(19) United States
(12) Patent Application Publication
(10) Pub. No.: US 2026/0055065 A1
(43) Pub. Date: Feb. 26, 2026

(54) PYRIDAZINONE COMPOUNDS WHICH MODULATE MUTANT PROTEINS FOR TREATING RESPIRATORY DISEASES

(71) Applicant: The Hospital for Sick Children, Toronto (CA)

(72) Inventors: Christine E Bear, Toronto (CA); Paul David William Eckford, Breslau (CA); Canhui Li, Toronto (CA); Mohabir Ramjeesingh, Toronto (CA); Ling-Jun Huan, Toronto (CA); Adrian Tanjala, Toronto (CA); Jia Xin Jiang, Toronto (CA); Daniel Vincent Paone, Lansdale, PA (US); Jakob Busch-Petersen, Riehen (CH)

(21) Appl. No.: 19/105,314

(22) PCT Filed: Aug. 24, 2023
(86) PCT No.: PCT/CA2023/051122

§ 371 Date: Feb. 20, 2025

Related U.S. Application Data
(60) Provisional application No. 63/400,857, filed on Aug. 25, 2022.

(51) Int. Cl. C07D 237/22; A61K 31/501; A61K 31/5377; A61P 11/00; C07D 401/04; C07D 403/04

(52) U.S. Cl. CPC C07D 237/22; A61K 31/501; A61K 31/5377; A61P 11/00; C07D 401/04; C07D 403/04

(57) ABSTRACT

The present disclosure relates to pyridazinone compound of the Formula (I) for the treatment of respiratory diseases, such as, chronic obstructive pulmonary disease (COPD), cystic fibrosis, cancer, Long QT syndrome or Dravet syndrome, which arise as a result of mis-folded or mis-shaped proteins.

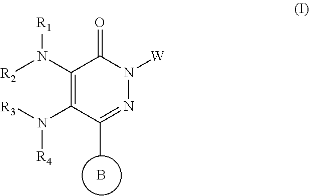

### CROSS-REFERENCE TO RELATED APPLICATION

This application claims the benefit of priority to U.S. Provisional Application No. 63/400,857, filed Aug. 25, 2022, the contents of which is incorporated herein by reference in its entirety.

### FIELD OF THE DISCLOSURE

This application relates to pyridazinone compounds of the Formula (I) for the treatment of respiratory diseases associated with mis-folded or mis-shaped proteins.

### BACKGROUND OF THE DISCLOSURE

Cystic fibrosis (CF), the most common fatal genetic disease among Canadians, is caused by a mutation in the cystic fibrosis transmembrane conductance regulator (CFTR/ABCC7) gene. This gene encodes the CFTR protein, a plasma membrane channel, which is responsible for chloride ion flux across the apical membrane of epithelial cells of hollow organs such as the lungs, pancreas, and digestive tract, and the skin (Gadsby, et al. (2006) The ABC protein turned chloride channel whose failure causes cystic fibrosis, Nature, 440: 477-483). This maintains the ion balance needed for a thin layer of mucus in these organs. Normally, after CFTR protein is synthesized in the endoplasmic reticulum, it is folded into its correct structural conformation, and then escorted into the Golgi apparatus for the addition of complex glycosylation and delivered to the plasma membrane.

CFTR is a member of the ATP-binding cassette (ABC) superfamily of membrane proteins, the members of which employ ATP hydrolysis to carry out biological processes, most commonly to actively transport substrates across membranes. CFTR is unique in that it is the only member of the family to be an ion channel; however, its architecture is similar to other ABC proteins, consisting of two membrane-spanning domains (MSDs), each linked to a nucleotide binding domain (NBD). A domain specific to CFTR is the regulatory (R) domain, which is structurally disordered and when phosphorylated, participates in channel gating. CFTR is also regulated by interdomain interactions as well as binding and hydrolysis at the ATP binding sites, located at the NBD1:NBD2 interface. Specifically, the ATP-driven dimerization of NBD1 and NBD2 allows the ion channel to open, while ATP hydrolysis dissociates the dimer and closes the gate (Serohijos, A. W. R., et al. (2007)).

The most common mutation, present in 70% of alleles, is ΔF508, a deletion (Δ) of the amino acid phenylalanine (Phe, F) at position 508 within CFTR protein (Welsh, M. J., et al. (1993), Dysfunction of CFTR bearing the delta F508 mutation. J. Cell Sci. Suppl., 17: 235-239). F508, located in NBD1 at its interface with MSD2, is thought to mediate the interdomain interaction for properfolding and channel function. Specifically, the aromatic side chain of F508 is thought to form an aromatic cluster with residues from the intracellular loop 4 (ICL4) of MSD2 and other residues from NBD1, playing an integral role in the stability of the tertiary structure of CFTR (Serohijos et al., supra.). The deletion of this crucial residue prevents CFTR from folding properly, and forces it to be retained in the endoplasmic reticulum, where it is targeted for degradation. In addition, this mutant has reduced function even when rescued to the cell surface. The deletion of F508 likely alters domain:domain interfaces in the protein, hindering proper channel gating. Overall, this mutation disrupts the extracellular ion balance, reducing hydration of the surface, leading to thick, sticky mucus on many vital organs such as the lungs and the pancreas that is readily susceptible to bacterial infection. Many other CFTR mutations are known, such as gating mutations, for example the G551 D mutation.

Small molecules can act as correctors, which promote forward trafficking of mutant CFTR protein to the cell surface, or as potentiators, which increase the channel activity of the mutant CFTR protein that has reached the plasma membrane.

Chronic obstructive pulmonary disease (COPD) is a disease syndrome characterized by airway inflammation induced by environmental toxins, primarily tobacco smoke and household and industrial air pollution. The presentation of COPD is quite heterogenous and believed to be a combination of disorders, including chronic bronchitis and emphysema, both of which cause diminished airflow at expiration and are associated with obstructed bronchi. Loss of CFTR channel function on the luminal surface of the airway epithelium is caused by environmental pollutants and this loss is thought to cause mucus obstruction.

### SUMMARY OF THE DISCLOSURE

The present application is directed to pyridazinone compounds of the Formula (I), and in one aspect of the disclosure, are useful for the treatment of respiratory diseases associated with mis-folded or mis-shaped proteins, such as cystic fibrosis. In one embodiment, the disease is COPD. In one embodiment, the compounds of the Formula (I) modulate mutant proteins having defects in activity and folding associated with respiratory diseases.

In one embodiment, the compounds of the Formula (I) have the following structure

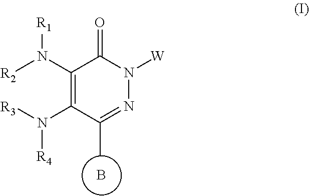

wherein
R₁ and R₂ are independently or simultaneously H or (C₁-C₆)-alkyl;
R₃ and R₄ are independently or simultaneously H or (C₁-C₆)-alkyl, or
R₃ and R₄ are joined together, with the nitrogen atom to which they are attached, to form a (C₅-C₆)-heteroaryl or (C₄-C₆)-heterocycloalkyl, each of which is optionally substituted with halo, OH, (C₁-C₆)-alkyl, or halogenated-(C₁-C₆)-alkyl;
Ring B is (C₆-C₁₀)-aryl or (C₅-C₁₀)-heteroaryl, each of which is optionally substituted with one or more of halo, OH, (C₁-C₆)-alkyl, halogenated-(C₁-C₆)-alkyl, (C₃-C₆)-cycloalkyl, or halogenated-(C₃-C₆)-cycloalkyl;
W is (C₁-C₆)-alkyl or —(C₀-C₆)-alkylene-(C₆-C₁₀)-aryl, each of which is optionally substituted with halo, OH, CN, (C₁-C₆)-alkyl, halogenated-(C₁-C₆)-alkyl, (C₁-C₆)-alkoxy, (C₃-C₆)-cycloalkyl, or halogenated-(C₃-C₆)-cycloalkyl;
   or any pharmaceutically acceptable salt, stereoisomer or solvate thereof.

The present disclosure also includes the use of a therapeutically effective amount of a compound of the Formula (I) as a potentiator, for example, in the treatment of cystic fibrosis.

The present disclosure also includes the use of a therapeutically effective amount of a compound of the Formula (I) as a potentiator, for example, in the treatment of COPD.

The present disclosure also includes a method for treating a patient with cystic fibrosis comprising administering a therapeutically effective amount of a compound of the Formula (I).

The present disclosure also includes a method for treating a patient with COPD comprising administering a therapeutically effective amount of a compound of the Formula (I).

In another embodiment, the compounds of the Formula (I) are co-administered with corrector compounds, whereby the corrector compounds are principally targeted at cellular processing errors and transporting the protein to the cell surface, while the potentiator compounds (of the Formula (I)) help to restore function of the protein, for example, by restoring cAMP-dependent chloride channel activity to misfolded proteins (such as CFTR) at the cell surface.

Other features and advantages of the present application will become apparent from the following detailed description. It should be understood, however, that the detailed description and the specific examples while indicating preferred embodiments of the application are given by way of illustration only, since various changes and modifications within the spirit and scope of the application will become apparent to those skilled in the art from this detailed description.

### BRIEF DESCRIPTION OF THE DRAWINGS

The disclosure is in the hereinafter provided paragraphs described, by way of example, in relation to the attached figures. The figures provided herein are provided for a better understanding of the example embodiments and to show more clearly how the various embodiments may be carried into effect. The figures are not intended to limit the present disclosure.

FIG. 1 shows micrographs in (A) and (B) of bead tracking and (C) a graph showing improved bead motility upon exposure of a compound of the disclosure.

FIG. 2 shows the dose responses for VX-770 and SK-POT (a compound of the disclosure) in potentiating cyclic AMP (forskolin activated) chloride channel activity by F508del-CFTR were measured in CFBE41o⁻ using the fluorescence based, membrane potential assay.

FIG. 3(i) shows cell-attached CFTR recordings. a—representative traces of recordings made in 150NMDG-CI base solution complemented with: b—1 mM forskolin, c—10 nM SK-POT analog, and d—10 mM Inhibitor 172 (1172); 0 current line represented by gray dashed line; the total number of CFTR channels in the cell-attached patch, which was used for representative traces shown, was estimated as 8. Vₘ=−80 mV; e—open probabilities for each drug normalized to open probability in presence of 1 uM forskolin (see Methods), (ii) Bar graph shows channel open probabilities normalized for total channel number in the presence of forskolin, forskolin plus SK-POT or following the addition of CFTR Inh-172 in the concentrations mentioned above. The error bars are standard deviations; n=5 for each drug.

FIG. 4(i) shows a graph of Representative Ussing chamber studies of SK-POT (1 μM) potentiation after forskolin activation (100 nM) in primary bronchial epithelial cultures. (ii). Bar graph shows forskolin-dependent changes in CFTR mediated short circuit with the addition of compound 11 or VX-770 (n=3 donors, n=2-3 technical replicates for each condition).

FIG. 5 shows top panel: Representative traces showing changes in forskolin stimulated (addition indicated as downward black arrow) CFTR dependent FLiPR signals measured in confluent Calu-3 cell cultures pre-exposed to cigarette smoke (CSE) and treated acutely with forskolin (+DMSO (VEH), solid circle). Addition of CFTR-inhibitor indicated with grey arrow. Superimposed, we show the effect of acute co-application of VX-770 (open squares) or SK-POT (open triangles) with forskolin. The bar graph (below) shows the inhibitory effect of cigarette smoke extract (−CSE vs. +CSE) on CFTR channel activity as measured using the FLiPR assay in Calu-3 monolayers (3 biological and 3-4 technical replicates). This bar graph also shows the relative impact of DMSO (VEH), VX-770 (770, 1 μM) or SK-POT, 1 μM) “p”-values determined using One-way Anova, Sidak's multiple comparison test. Only SK-POT rescued CFTR channel function in the presence of cigarette smoke extract.

FIG. 6(i-iii) show traces of motile green microspheres over a 5-second period, captured at 10 frames-per-second at 20× objective magnification. Cells were chronically pre-treated for 24 hours with 2% cigarette smoke extract plus DMSO (VEH), VX-770 or SK-POT and then acutely treated with 10 μM forskolin (FSK) for 20 minutes at room temperature prior to recording. ii. Traces of motile green microspheres under the same conditions as in i., except 1 μM ivacaftor (VX-770) co-applied during the chronic treatment. Dot colour within the trace represents the point along the 5-second period, with violet (cooler) representing the beginning and red/warmer colours representing the end of the period. iv. Aggregate results of mean bead velocity across n=4 donors, comparing velocities without CSE to velocities with CSE (with or without chronic co-application of VEH, VX-770 (iv) or SK-POT (v)). Points represent the mean bead velocities of individual videos, with n=4-6 videos obtained per donor and condition. Horizontal lines represent mean of all videos, and error bars represent standard deviation. Statistical significance assessed by one-way ANOVA with the Sidak's multiple comparison test.

FIG. 7 shows (a) Representative Ussing chamber studies of SK-POT (1 μM) potentiation after forskolin activation (100 nM) in primary bronchial epithelial cultures prepared from adult ferret trachea. (b) Bar graph shows forskolin-dependent changes in CFTR mediated short circuit with the addition of SK-9919 or VX-770 (n=3 donors, n=2-3 technical replicates for each condition). Student's “t” test was conducted.

### DETAILED DESCRIPTION OF THE INVENTION

### (I) Definitions

The term “(C₁-Cₙ)-alkyl” as used herein means straight and/or branched chain, saturated alkyl radicals containing from one to “n” carbon atoms and includes (depending on the identity of n) methyl, ethyl, propyl, isopropyl, n-butyl, s-butyl, isobutyl, t-butyl, 2,2-dimethylbutyl, n-pentyl, 2-methylpentyl, 3-methylpentyl, 4-methylpentyl, n-hexyl and the like, where the variable n is an integer representing the largest number of carbon atoms in the alkyl radical.

The term “(C₁-Cₙ)-alkoxy” as used herein means straight and/or branched chain, saturated alkoxy radicals containing from one to “n” carbon atoms and includes (depending on the identity of n) methoxy, ethoxy, propoxy, isopropoxy, n-butoxy, s-butoxy, isobutoxy, t-butoxy, pentoxy, hexoxy, and the like, where the variable n is an integer representing the largest number of carbon atoms in the alkyl radical.

The term “(C₃-C₆)-cycloalkyl” as used herein means a monocyclic saturated or partially unsaturated carbocylic group containing from 3 to 6 carbon atoms and includes (depending on the identity of m) cyclopropyl, cyclobutyl, cyclopentyl, cyclohexyl, cyclohexenyl, and the like.

The term “heterocycloalkyl” as used herein means a monocyclic saturated or partially unsaturated group containing, for example, from 5 to 6 ring atoms and includes one, two, three, or four are heteromoieties independently selected from N, NH, N(C₁₋₆alkyl), O and S, pyrrolidinyl, piperidinyl, piperazinyl, oxazolidinyl, isoxazolidinyl, morpholinyl, thiazolidinyl, isothiazolidinyl, and tetrahydrofuryl.

The term “aryl” as used herein means a monocyclic or bicyclic aromatic ring system containing at least one aromatic ring and from 6 to 10 carbon atoms and includes phenyl, naphthyl, 1,2-dihydronaphthyl, 1,2,3,4-tetrahydronaphthyl, indanyl, indenyl and the like.

The term “heteroaryl” as used herein means a monocyclic or bicyclic ring system containing one or two aromatic rings and from 5 to 10 atoms and includes one, two, three, or four are heteromoieties independently selected from N, NH, N(C₁₋₆alkyl), O and S and includes thienyl, thiazolyl, furyl, pyrrolyl, pyrididyl, indolyl, quinolyl, isoquinolyl, tetrahydroquinolyl, benzofuryl, benzothienyl and the like.

The term “halo” as used herein means halogen and includes chloro, fluoro, bromo, iodo and the like.

The term “halogenated” as used herein, for example with reference to halogenated-(C₁-C₆)-alkyl, means that at least one, including all, of the hydrogens on the referenced group is replaced with a halogen atom.

The term “stereoisomer” as used herein means an isomer that possesses identical constitution as a corresponding stereoisomer, but which differs in the arrangement of its atoms in space from the corresponding stereoisomer. For example, stereoisomers may be enantiomers, diastereomers and/or cis-trans (E/Z) isomers. It should be understood that the compounds of formula (I) may comprise single enantiomers, single diastereomers as well as mixtures thereof at any ratio (for example racemic mixtures, non-racemic mixtures).

The term “solvate” as used herein means a pharmaceutically acceptable solvate form of a specified compound of the Formula (I) that retains the biological effectiveness of such compound, for example, resulting from a physical association of the compound with one or more solvent molecules. Examples of solvates, without limitation, include compounds of the invention in combination with water, 1-propanol, 2-propanol, ethanol, methanol, DMSO, ethyl acetate, acetic acid, or ethanolamine. When the solvent is water, the form is known as a “hydrate”.

The term “therapeutically effective amount” as used herein means a dosage to produce a selected effect. For example, an effective amount of a compound of Formula (I) is an amount which is sufficient for the compound to act as a potentiator, and for example, ameliorate or improve cystic fibrosis or COPD, or the symptoms thereof.

The term “pharmaceutically-acceptable salt” refers to salts that retain the biological effectiveness and properties of the compounds of the Formula (I) and which are not biologically or otherwise undesirable. In many cases, the disclosed compounds are capable of forming acid or base salts by virtue of the presence of acidic or basic moieties. The preparation of the salts and suitable acids or bases is known in the art.

### (II) Compounds of the Formula (I)

The present disclosure relates to pyridazinone compounds of the Formula (I), and in one aspect of the disclosure, are useful for the treatment of diseases associated with mis-filed or mis-shaped proteins. In one embodiment, the compounds of the Formula (I) have the following structure

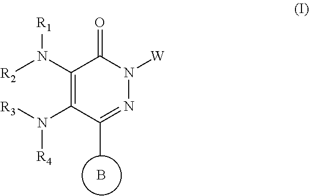

wherein
R₁ and R₂ are independently or simultaneously H or (C₁-C₆)-alkyl;
R₃ and R₄ are independently or simultaneously H or (C₁-C₆)-alkyl, or
R₃ and R₄ are joined together, with the nitrogen atom to which they are attached, to form a (C₅-C₆)-heteroaryl or (C₄-C₆)-heterocycloalkyl, each of which is optionally substituted with halo, OH, (C₁-C₆)-alkyl, or halogenated-(C₁-C₆)-alkyl;
Ring B is (C₆-C₁₀)-aryl or (C₅-C₁₀)-heteroaryl, each of which is optionally substituted with one or more of halo, OH, (C₁-C₆)-alkyl, halogenated-(C₁-C₆)-alkyl, (C₃-C₆)-cycloalkyl, or halogenated-(C₃-C₆)-cycloalkyl;
W is (C₁-C₆)-alkyl or —(C₀-C₆)-alkylene-(C₆-C₁₀)-aryl, each of which is optionally substituted with one or more of halo, OH, CN, (C₁-C₆)-alkyl, halogenated-(C₁-C₆)-alkyl, (C₁-C₆)-alkoxy, (C₃-C₆)-cycloalkyl, or halogenated-(C₃-C₆)-cycloalkyl;
   or any pharmaceutically acceptable salt, stereoisomer or solvate thereof.

In one embodiment, R₁ and R₂ are independently or simultaneously H or (C₁-C₃)-alkyl. In another embodiment, R₁ and R₂ are independently or simultaneously H or CH₃. In another embodiment, R₁ and R₂ are H.

In another embodiment, R₃ and R₄ are independently or simultaneously H or (C₁-C₃)-alkyl, or R₃ and R₄ are joined together, with the nitrogen atom to which they are attached, to form a (C₅)-heteroaryl or (C₄-C₆)-heterocycloalkyl, each of which is optionally substituted with halo, OH, (C₁-C₃)-alkyl, or halogenated-(C₁-C₃)-alkyl.

In another embodiment, R₃ and R₄ are independently or simultaneously H or CH₃. In another embodiment, R₃ and R₄ are CH₃.

In another embodiment, R₃ and R₄ are joined together, with the nitrogen atom to which they are attached, to form optionally substituted piperidinyl, pyrrolidinyl, morpholinyl, azetidinyl, or pyrazolyl.

In another embodiment, Ring B is (C₆)-aryl or (C₅-C₆)-heteroaryl, each of which is optionally substituted with one or more of halo, OH, (C₁-C₆)-alkyl, halogenated-(C₁-C₆)-alkyl, (C₃-C₆)-cycloalkyl, or halogenated-(C₃-C₆)-cycloalkyl. In another embodiment, Ring B is phenyl or pyridinyl, optionally substituted with one or more of halo, (C₁-C₆)-alkyl, or halogenated-(C₁-C₆)-alkyl. In another embodiment, Ring B is phenyl or pyridinyl, optionally substituted with one or more of fluoro or trifluoro-methyl. In a further embodiment, Ring B has the following structure:

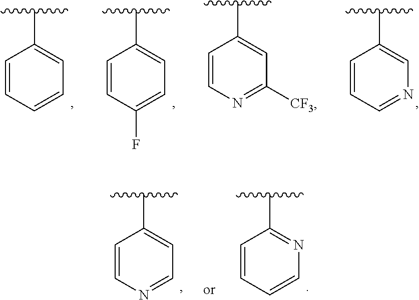

In another embodiment, W is (C₁-C₄)-alkyl or —(C₀-C₄)-alkylene-(C₆-C₁₀)-aryl, each of which is optionally substituted with halo, OH, CN, (C₁-C₄)-alkyl, halogenated-(C₁-C₄)-alkyl, (C₁-C₄)-alkoxy, (C₃-C₆)-cycloalkyl, or halogenated-(C₃-C₆)-cycloalkyl. In another embodiment, W is (C₁-C₄)-alkyl or —(C₀-C₁)-alkylene-phenyl, each of which is optionally substituted with one or more of halo, OH, CN, (C₁-C₄)-alkyl, halogenated-(C₁-C₄)-alkyl, or (C₁-C₄)-alkoxy. In another embodiment, W has the following structure

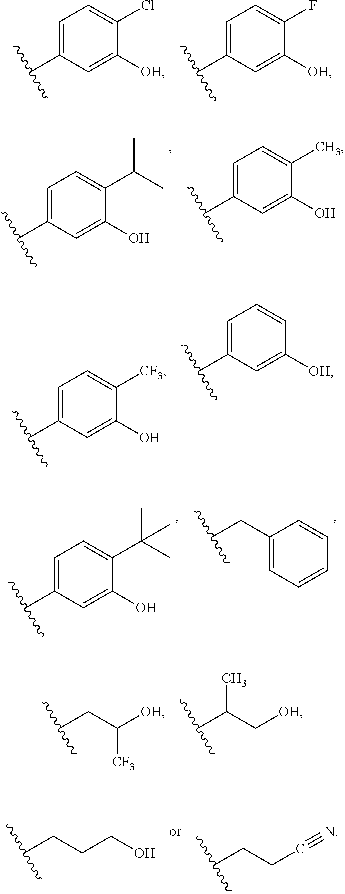

In one embodiment of the disclosure, the compound of Formula (I) is

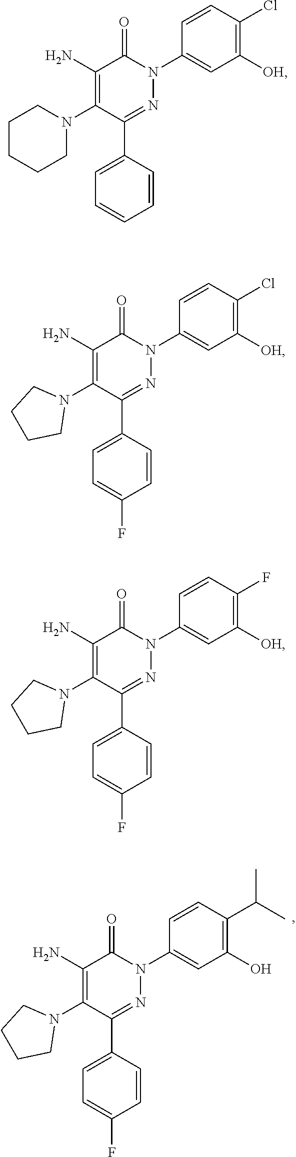 [SMILES: Nc1c(N2CCCCC2)c(-c2ccccc2)nn(-c2ccc(Cl)c(O)c2)c1=O ; Nc1c(N2CCCC2)c(-c2ccc(F)cc2)nn(-c2ccc(Cl)c(O)c2)c1=O ; Nc1c(N2CCCC2)c(-c2ccc(F)cc2)nn(-c2ccc(F)c(O)c2)c1=O ; CC(C)c1ccc(-n2nc(-c3ccc(F)cc3)c(N3CCCC3)c(N)c2=O)cc1O]

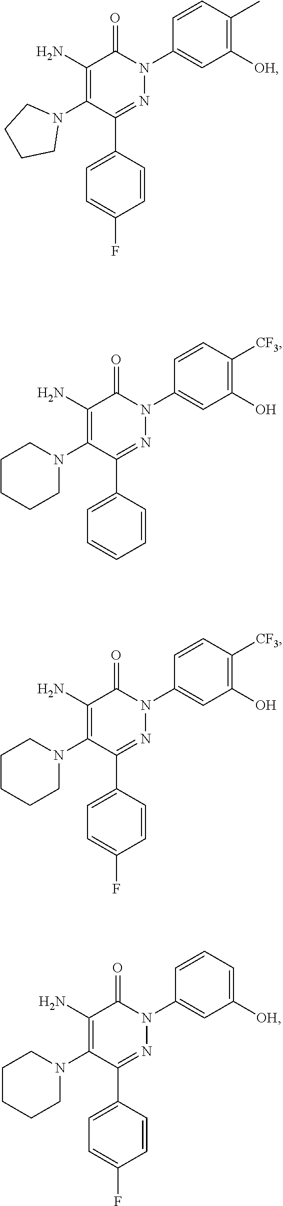 [SMILES: Cc1ccc(-n2nc(-c3ccc(F)cc3)c(N3CCCC3)c(N)c2=O)cc1O ; Nc1c(N2CCCCC2)c(-c2ccccc2)nn(-c2ccc(C(F)(F)F)c(O)c2)c1=O ; Nc1c(N2CCCCC2)c(-c2ccc(F)cc2)nn(-c2ccc(C(F)(F)F)c(O)c2)c1=O ; Nc1c(N2CCCCC2)c(-c2ccc(F)cc2)nn(-c2cccc(O)c2)c1=O]

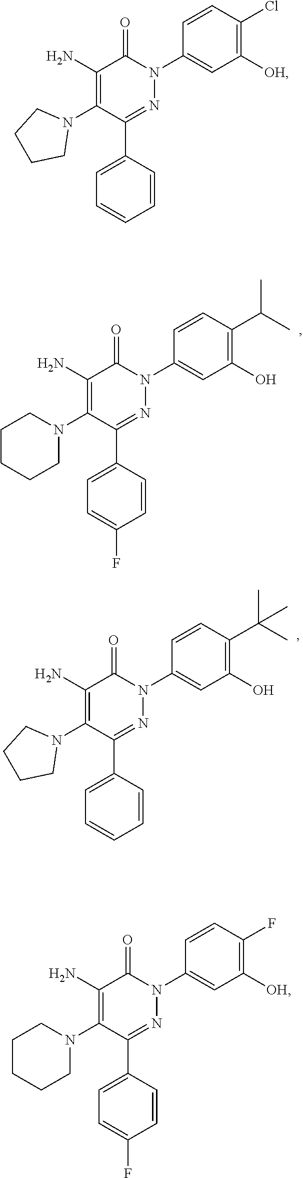 [SMILES: Nc1c(N2CCCC2)c(-c2ccccc2)nn(-c2ccc(Cl)c(O)c2)c1=O ; CC(C)c1ccc(-n2nc(-c3ccc(F)cc3)c(N3CCCCC3)c(N)c2=O)cc1O ; CC(C)(C)c1ccc(-n2nc(-c3ccccc3)c(N3CCCC3)c(N)c2=O)cc1O ; Nc1c(N2CCCCC2)c(-c2ccc(F)cc2)nn(-c2ccc(F)c(O)c2)c1=O]

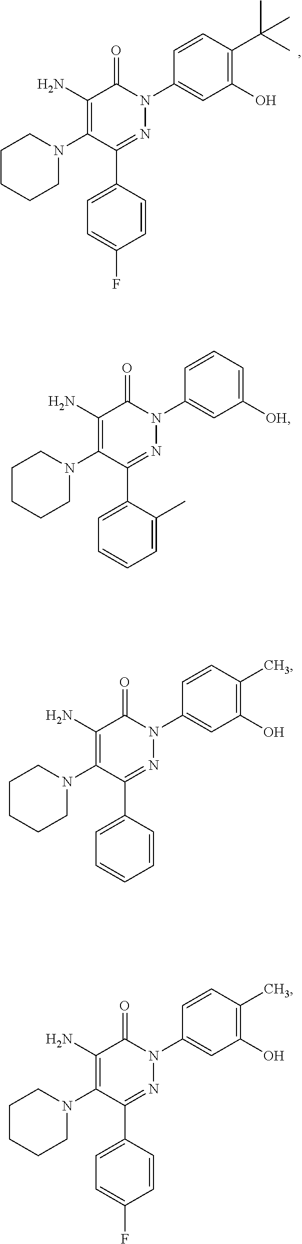 [SMILES: CC(C)(C)c1ccc(-n2nc(-c3ccc(F)cc3)c(N3CCCCC3)c(N)c2=O)cc1O ; Cc1ccccc1-c1nn(-c2cccc(O)c2)c(=O)c(N)c1N1CCCCC1 ; Cc1ccc(-n2nc(-c3ccccc3)c(N3CCCCC3)c(N)c2=O)cc1O ; Cc1ccc(-n2nc(-c3ccc(F)cc3)c(N3CCCCC3)c(N)c2=O)cc1O]

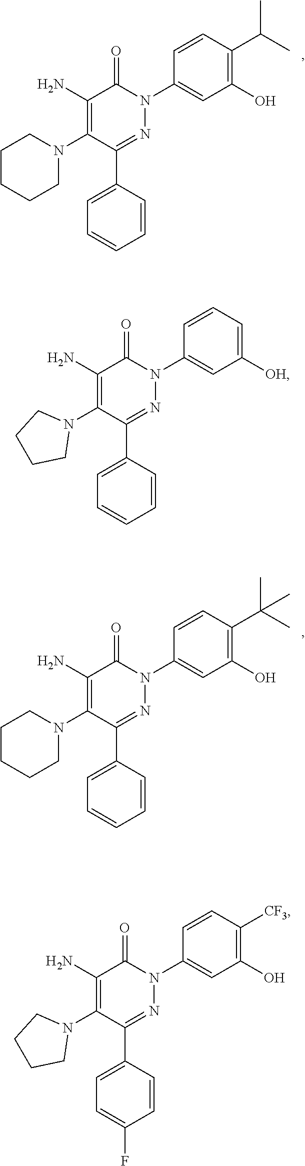 [SMILES: CC(C)c1ccc(-n2nc(-c3ccccc3)c(N3CCCCC3)c(N)c2=O)cc1O ; Nc1c(N2CCCC2)c(-c2ccccc2)nn(-c2cccc(O)c2)c1=O ; CC(C)(C)c1ccc(-n2nc(-c3ccccc3)c(N3CCCCC3)c(N)c2=O)cc1O ; Nc1c(N2CCCC2)c(-c2ccc(F)cc2)nn(-c2ccc(C(F)(F)F)c(O)c2)c1=O]

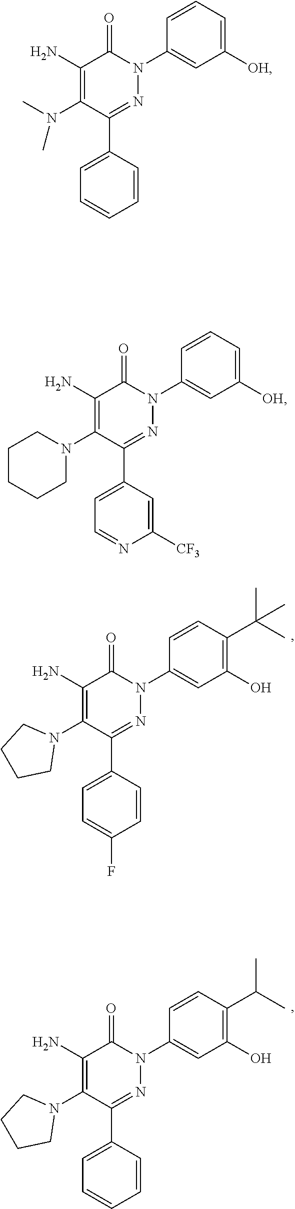 [SMILES: CN(C)c1c(-c2ccccc2)nn(-c2cccc(O)c2)c(=O)c1N ; Nc1c(N2CCCCC2)c(-c2ccnc(C(F)(F)F)c2)nn(-c2cccc(O)c2)c1=O ; CC(C)(C)c1ccc(-n2nc(-c3ccc(F)cc3)c(N3CCCC3)c(N)c2=O)cc1O ; CC(C)c1ccc(-n2nc(-c3ccccc3)c(N3CCCC3)c(N)c2=O)cc1O]

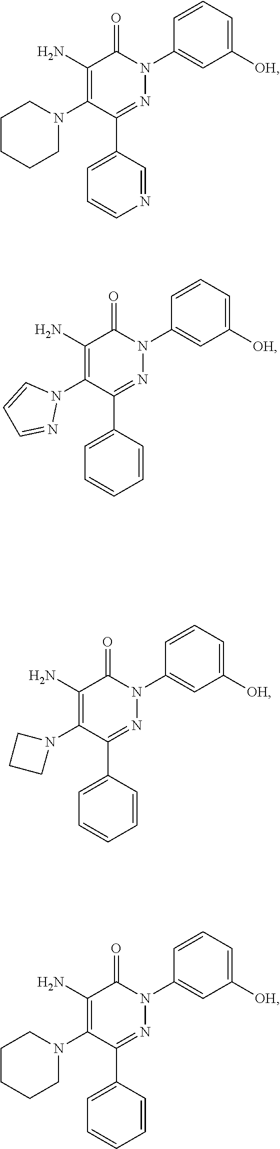 [SMILES: Nc1c(N2CCCCC2)c(-c2cccnc2)nn(-c2cccc(O)c2)c1=O ; Nc1c(-n2cccn2)c(-c2ccccc2)nn(-c2cccc(O)c2)c1=O ; Nc1c(N2CCC2)c(-c2ccccc2)nn(-c2cccc(O)c2)c1=O ; Nc1c(N2CCCCC2)c(-c2ccccc2)nn(-c2cccc(O)c2)c1=O]

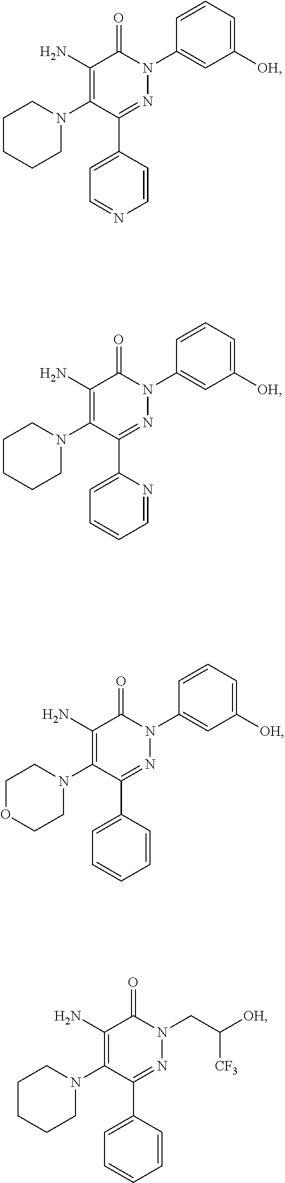 [SMILES: Nc1c(N2CCCCC2)c(-c2ccncc2)nn(-c2cccc(O)c2)c1=O ; Nc1c(N2CCCCC2)c(-c2ccccn2)nn(-c2cccc(O)c2)c1=O ; Nc1c(N2CCOCC2)c(-c2ccccc2)nn(-c2cccc(O)c2)c1=O ; Nc1c(N2CCCCC2)c(-c2ccccc2)nn(CC(O)C(F)(F)F)c1=O]

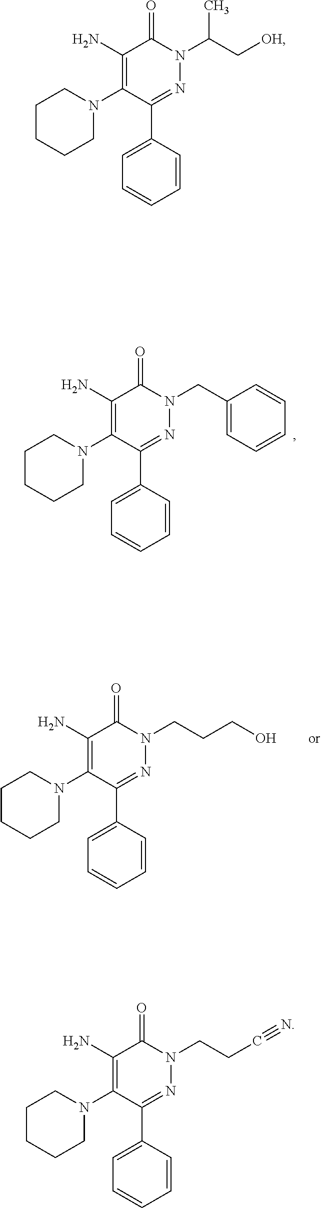 [SMILES: CC(CO)n1nc(-c2ccccc2)c(N2CCCCC2)c(N)c1=O ; Nc1c(N2CCCCC2)c(-c2ccccc2)nn(Cc2ccccc2)c1=O ; Nc1c(N2CCCCC2)c(-c2ccccc2)nn(CCCO)c1=O ; N#CCCn1nc(-c2ccccc2)c(N2CCCCC2)c(N)c1=O]

In one embodiment, the compound is

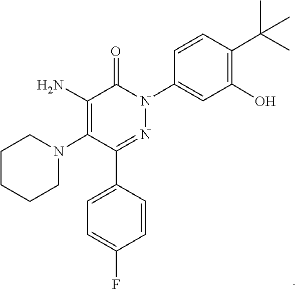 [SMILES: CC(C)(C)c1ccc(-n2nc(-c3ccc(F)cc3)c(N3CCCCC3)c(N)c2=O)cc1O]

The present disclosure also includes pharmaceutical compositions comprising a compound of the Formula (I) as defined above (compounds of the disclosure), or pharmaceutically acceptable salts, solvates, and prodrugs thereof, and a pharmaceutically acceptable carrier or diluent. The compounds are suitably formulated into pharmaceutical compositions for administration to subjects, preferably humans in a biologically compatible form suitable for administration in vivo.

The compositions containing the compounds of disclosure can be prepared by known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (2003-20th edition) and in The United States Pharmacopeia: The National Formulary (USP 24 NF19) published in 1999. On this basis, the compositions include, albeit not exclusively, solutions of the substances in association with one or more pharmaceutically acceptable vehicles or diluents, and contained in buffered solutions with a suitable pH and iso-osmotic with the physiological fluids.

The compounds of disclosure may be used pharmaceutically in the form of the free base, in the form of salts, solvates and as hydrates. All forms are within the scope of the disclosure. Acid and basic addition salts may be formed with the compounds of the disclosure for use as sources of the free base form even if the particular salt per se is desired only as an intermediate product as, for example, when the salt is formed only for the purposes of purification and identification. All salts that can be formed with the compounds of the disclosure are therefore within the scope of the present disclosure.

In one embodiment, the compounds of the Formula (I) of the present disclosure are formulated into pharmaceutical compositions in a manner which will be familiar to any person skilled in the art by bringing the compound of Formula (I), together with suitable, non-toxic, inert, therapeutically compatible solid, liquid or aerosol carrier materials and, if desired, usual pharmaceutical adjuvants.

Suitable carrier materials are not only inorganic carrier materials, but also organic carrier materials. Suitable carrier materials for topical preparations are glycerides, semi-synthetic and synthetic glycerides, hydrogenated oils, liquid waxes, liquid paraffins, liquid fatty alcohols, sterols, polyethylene glycols and cellulose derivatives.

Usual stabilizers, preservatives, wetting and emulsifying agents, consistency-improving agents, salts for varying the osmotic pressure, buffer substances, solubilizers, colorants and antioxidants come into consideration as pharmaceutical adjuvants.

In some embodiments, the present disclosure includes compositions comprising a compound of the Formula (I) as a potentiator, and another active agent which is a corrector for the treatment of diseases associated with mis-folded and/or mis-shaped proteins. In one embodiment, the corrector compound is VRT-534 or VX-809.

In another embodiment, when the compound of Formula (I) is co-administered with a corrector, the active agents (compound of the Formula (I) and corrector) may be administered simultaneously or consecutively.

### (III) Methods of Medical Treatment

The present disclosure includes methods of medical treatment comprising the administration of a compound of Formula (I) to a mammal. In some embodiments, the present disclosure includes methods for the treatment of respiratory diseases which arise as a result of mis-folded and/or mis-shaped proteins. In one embodiment, the disclosure includes methods for modulating mutant proteins having defects in activity and/or folding associated with respiratory diseases comprising administering compounds of the Formula (I). In one embodiment, the respiratory disease is cystic fibrosis or chronic obstructive pulmonary disease (COPD). In another embodiment, the disclosure includes methods for the treatment of other diseases associated with mis-folded or mis-shaped proteins, such as Long QT syndrome or Dravet syndrome.

In one embodiment, the present disclosure includes a method for treating a patient with a respiratory disease which arises as a result of mis-folded or mis-shaped proteins comprising administering a therapeutically effective amount of a compound of Formula (I). In one embodiment, the present disclosure includes a method for treating a patient with cystic fibrosis comprising administering a therapeutically effective amount of a compound of Formula (I). In another embodiment, the cystic fibrosis is a result of the ΔF508 mutation in the CFTR protein.

In one embodiment, the present disclosure includes a method for treating a patient with a respiratory disease which arises as a result of mis-folded or mis-shaped proteins comprising administering a therapeutically effective amount of a compound of Formula (I). In one embodiment, the present disclosure includes a method for treating a patient with COPD comprising administering a therapeutically effective amount of a compound of Formula (I). In another embodiment, the COPD is a result of acquired CF where the CFTR protein function is reduced by cigarette smoke or other environmental toxins.

In another embodiment of the disclosure, there is also included a method of treating diseases which arise as a result of mis-folded or mis-shaped proteins comprising administering to a subject, such as a human, a therapeutically effective amount of a compound of Formula (I). In one embodiment, the disclosure includes methods for modulating mutant proteins having defects in activity and/or folding associated with respiratory diseases comprising administering compounds of the Formula (I). In another embodiment of the disclosure, there is also included a method of treating Long QT syndrome comprising administering, to a subject, such as a human, a therapeutically effective amount of a compound of Formula (I). In another embodiment of the disclosure, there is also included a method of treating Dravet syndrome (epilepsy), comprising administering, to a subject, such as a human, a therapeutically effective amount of a compound of Formula (I). In another embodiment of the disclosure, there is also included a method of treating cancer associated with mis-folded or mis-shaped proteins, such as the p53 protein, comprising administering, to a subject, such as a human, a therapeutically effective amount of a compound of Formula (I).

In other embodiments, the present disclosure also includes a use of the compounds of the Formula (I) for the treatment of respiratory diseases which arise as a result of mis-folded or mis-shaped proteins. In one embodiment, the respiratory disease is cystic fibrosis. In one embodiment, the disease is COPD.

The dosage of a compound of Formula (I) varies within wide limits depending on the disease to be controlled, the age and the individual condition of the patient and the mode of administration, and will, of course, be fitted to the individual requirements in each particular case. For adult patients a daily dosage of about 1 mg to about 1000 mg, especially about 1 mg to about 100 mg, comes into consideration. Depending on the dosage it is convenient to administer the daily dosage in several dosage units.

Although the disclosure has been described in conjunction with specific embodiments thereof, if is evident that many alternatives, modifications and variations will be apparent to those skilled in the art. Accordingly, it is intended to embrace all such alternatives, modifications and variations that fall within the spirit and broad scope of the appended claims. In addition, citation or identification of any reference in this application shall not be construed as an admission that such reference is available as prior art to the present disclosure.

### EXAMPLES

The operation of the disclosure is illustrated by the following representative examples. As is apparent to those skilled in the art, many of the details of the examples may be changed while still practicing the disclosure described herein.

### Materials and Methods

Cell culture: HEK293 cells stably expressing human deltaF508 CFTR were cultured in DMEM/F12 medium containing 10% fetal bovine serum (FBS) and 600 micrograms/mL geneticin. For functional CFTR assays, cryo-preserved cells were quickly thawed in a 37° C. water bath, diluted with warm DMEM/F12 medium containing 10% FBS, spun down at 300×g for 5 mins and resuspended in the same medium at a density of 300,000 cells/mL. A cystic fibrosis bronchial-derived cell line complemented with a 4.7 Kb deltaF508 CFTR cDNA (“CFBE”) was obtained from Dr. Dieter Gruenert under a license from UCSF. These cells were cultured in MEM with Earle's salt (Invitrogen)supplemented with 10% fetal bovine serum (Invitrogen) and 300 micrograms/mL hygromycin (Millipore). CFBE cells were grown on flasks coated with a fibronectin solution containing LHC basal medium (Invitrogen), 0.001% bovine serum albumin (Sigma), 1% v/v Vitrogen 100 (BD Bioscience) and 10 micrograms/mL human fibronectin (Life Technologies). All cells were grown at 37° C. and 5% CO₂ unless specified for an assay.

HEK293 deltaF508 CFTR cell potentiator assay: Cells were plated in 384-well black, poly-D-Lysine-coated plates (Greiner) at a density of 15,000 cells per well and placed in a 37° C., 5% CO₂ incubator for 2-3 hours. The cell plates were then placed in a 30° C., 5% CO₂ incubator for 20 to 24 hours to allow temperature rescue of delF508-CFTR expression. Prior to the assay, the cell medium was removed and 20 microliters/well of assay buffer containing blue membrane potential dye (10 ml diluted dye per 200 ml assay buffer, Molecular Devices) were added. The assay buffer was a modified Tyrode's buffer containing 140 mM sodium gluconate, 0.5 mM potassium gluconate, 2 mM calcium gluconate, 2 mM magnesium gluconate, 10 mM HEPES, 12 mM NaHCO₃, pH7.4. Cells were then incubated with this buffer for 45 to 60 mins in a 30° C., 5% CO₂ incubator. DelF508-CFTR activity was then measured on a FLIPR fluorescence plate reader (Molecular Devices). Activity was triggered with the addition of 10 microliters/well of assay buffer containing 90 nM genistein (Sigma) and compounds, 90 nM genistein and DMSO (1.6%, negative control) or 90 nM genistein plus 30 micromolar forskolin (positive control, Sigma). Changes in fluorescence were measured with the following filter settings, excitation wavelength: 510-545 nm, emission wavelength: 565-625 nm.

HEK293 deltaF508 CFTR cell corrector assay: Cells were plated in 384-well black, poly-D-Lysine-coated plates (Greiner) containing 0.5 microliter per well of DMSO-diluted compounds, DMSO alone (negative control), or mixed with 1 micromolar VX-661 (positive control, Selleckchem) at a density of 15,000 cells per well and placed in a 37° C., 5% CO₂ incubator for 18 to 24 hours. Prior to the assay, the cell medium was removed and 20 microliters/well of modified Tyrode's assay buffer containing blue membrane potential dye were added. Cells were incubated with this buffer for 45 to 60 mins in a 37° C., 5% CO₂ incubator. DelF508-CFTR activity was then measured on a FLIPR. Activity was triggered with the addition of 10 microliters/well of assay buffer containing 30 micromolar genistein and 30 micromolar forskolin. Changes in fluorescence were measured as described above.

CFBE potentiator assay: CFBE cells were dissociated for 10 mins at 37° C. with Hank's balanced salt solution containing 0.6 mM EDTA and 10% of a 0.25% Trypsin/EDTA solution (all from Life Technologies). An equal amount of CFBE cell growth medium (see Cell culture above) was then added to the flasks and cells were spun at 500×g for 10 mins. Cells were re-suspended in cell medium at a density of 150,000 cells/mL and plated in 384-well black, poly-D-Lysine-coated plates (7,500 cells/well). Cells were then grown at 37° C., 5% CO₂ for a total of 6-8 days to allow differentiation. Cell medium was changed every 2-3 days. The day before each experiment, culture medium was removed, fresh culture medium was added and cells were placed at 30° C., 5% CO₂ for 18-24 hrs to allow temperature rescue of delF508 CFTR. Potentiation activity was measured as described above for HEK cells except cells were stained for exactly 60 mins, compounds (or DMSO for negative control) were mixed with assay buffer containing 3 micromolar forskolin, and the positive control wells had assay buffer containing 3 micromolar forskolin and 9 micromolar VX-770 (Selleckchem). The results are shown in Table 1.

CFBE corrector assay: CFBE cells were dissociated and grown for 6-8 days in 384-well plates as described above. The day before each experiment, cell growth medium was removed, 50 microliters of medium containing compounds, 0.1% DMSO (negative control), or 1 micromolar VX-809 (positive control, Selleckchem) were added, and cells were placed at 30° C., 5% CO₂ for 18-24 hrs to allow temperature rescue of delF508 CFTR. Correction activity was measured as described above for HEK cells except cells were stained for exactly 60 mins and activity was triggered by addition of 10 microliters/well of assay buffer containing 3 micromolar forskolin and 9 micromolar VX-770.

Data analysis: CFTR activity was defined as maximum fluorescence signal after addition minus baseline fluorescence. All data were normalized to each plate's positive and negative controls and expressed as percent responses. Concentration response curves were analyzed by fitting the data to a 4-parameter logistic equation in Microcal's Origin or IDBS' Activity Base software.

### Example 1—Synthesis of Intermediate A

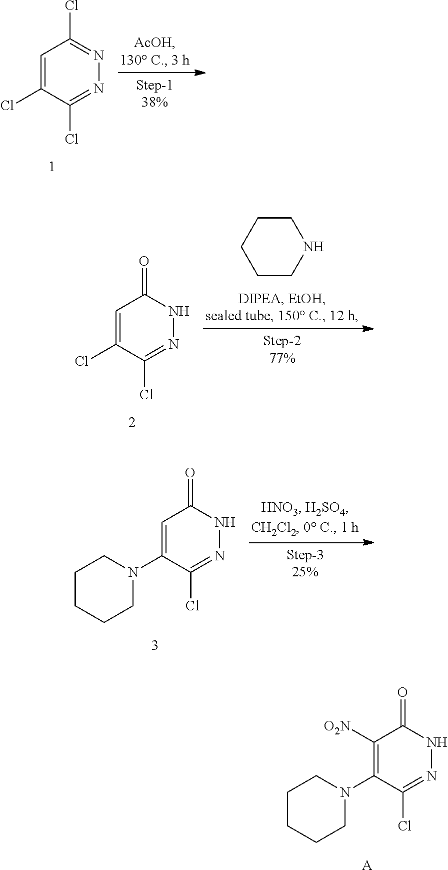

### Synthesis of 5,6-dichloropyridazin-3(2H)-one (2)

A stirring solution of 3,4,6-trichloropyridazine (1) (30.0 g, 163.4 mmol) in glacial acetic acid (120 mL) was heated to reflux temperature (130° C.). After 3 h stirring, the reaction mixture was cooled to RT and poured into ice-cold water (500 mL). The solid precipitated was filtered, washed with ice-cold water (50 mL×2) and dried under vacuum to afford 2 (10.3 g, 38%) as an off-white solid. ¹HNMR (400 MHz, CDCl₃): δ 11.19 (bs, 1H), 7.16 (s, 1H); LCMS (ESI): m/z 162.9 [M−H⁺]; 95.3%; RT=1.92 min (XBrigde C18 column, 5 mM ammonium bicarbonate in water with MeCN.)

### Synthesis of 6-chloro-5-(piperidin-1-yl)pyridazin-3(2H)-one (3)

A reaction tube was charged with 2 (25.0 g, 151.6 mmol), piperidine (26.9 mL, 272.0 mmol), disiopropylethylamine (132.5 ml, 757.6 mmol) and ethanol (125 mL) at RT under inert atmosphere. The reaction tube was capped and stirred for 12 h at 150° C. After complete consumption of starting material (monitored by TLC), the reaction mixture was cooled to RT and concentrated under reduced pressure. The residue obtained was diluted with water (250 mL) and extracted with EtOAc (2×250 mL). The combined organic layers were washed with 10% aqueous HCl solution (50 mL), dried over anhydrous Na₂SO₄ and concentrated under reduced pressure. The crude product was triturated with pentane (100 mL) to afford 3 (25 g, 77%) as a brown solid. ¹HNMR (400 MHz, DMSO-d₆): δ 12.68 (brs, 1H; D₂O exchangeable), 6.13 (s, 1H), 2.93-3.11 (m, 4H), 1.44-1.75 (m, 6H); LCMS (ESI): m/z 214.1 [M+H⁺]; 98.3%; RT=1.81 min (Acquity BEH C18 column, 0.1% formic acid/MeCN with 0.1% formic acid.

### Synthesis of 6-chloro-4-nitro-5-(piperidin-1-yl)pyridazin-3(2H)-one (A)

To a stirred solution of 3 (3.0 g, 14.04 mmol) in CH₂Cl₂ (50 mL), was added concentrated H₂SO₄ (18 mL) followed by fuming nitric acid (5 mL) at 0° C. and stirred for 1 h. Reaction was monitored by TLC. The reaction mixture was diluted with ice-cold water (200 mL) and extracted with CH₂Cl₂ (2×100 mL). The combined organic layer was dried over anhydrous Na₂SO₄ and concentrated under reduced pressure. Note: The reaction was repeated in four batches in parallel and the obtained crude product was combined and purified through silica gel column chromatography using 20% EtOAc in petroleum ether to afford A (4.6 g, 25%) as a yellow solid. ¹HNMR (400 MHz, CDCl₃): δ 10.74 (br s, 1H), 3.26-3.21 (m, 4H), 1.80-1.65 (m, 6H); LCMS (ESI): m/z 257.1 [M−H⁺]; 93.0%; RT=2.98 min (Acquity BEH C18 column, 0.05% formic acid/MeCN with 0.05% formic acid.

### Example 2—Synthesis of Intermediate B

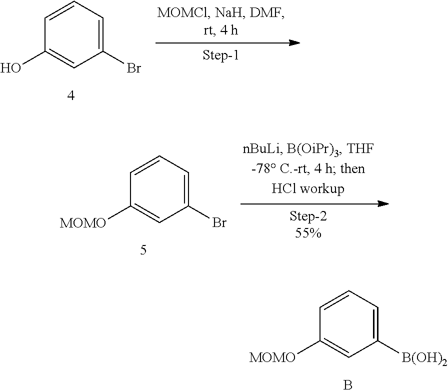

### Synthesis of 1-bromo-3-(methoxymethoxy)benzene (5)

To a stirred solution of 3-bromophenol (4) (20.0 g, 115.6 mmol) in DMF (300 mL), were added NaH (5.1 g, 127.5 mmol; 60% in mineral oil) followed by MOM-chloride (9.66 mL, 127.18 mmol) at 0° C. under inert atmosphere. The reaction was stirred at room temperature for 4 h. The reaction was monitored by TLC. The reaction mixture was diluted with ice-cold water (250 mL) and extracted with EtOAc (150 mL). The combined organic layer was dried over anhydrous Na₂SO₄ and concentrated under reduced pressure to afford 5 (25.0 g) as a brown gum, which was directly taken for next reaction without purification. ¹HNMR (400 MHz, CDCl₃): δ 7.24-7.20 (m, 1H), 7.16-7.11 (m, 2H), 6.99-6.94 (m, 1H), 5.16 (s, 2H), 3.47 (s, 3H).

### Synthesis of (3-(methoxymethoxy)phenyl)boronic acid (B)

To a stirred solution of 5 (25.0 g, 115.17 mmol) in THE (250 mL), was added n-butyllithium (50.8 mL; 126.7 mmol; 2.5M solution in hexane) drop wise at −78° C. and stirred at same temperature for 1 h. To this, triisopropyl borate (30 mL, 138.21 mmol) was added drop wise at −78° C. and maintained at room temperature for 4 h. After completion of reaction (monitored by TLC), the reaction mixture was quenched with 1N aqueous HCl solution (40 mL) and extracted with EtOAc (150 mL). The organic layer was dried over anhydrous Na₂SO₄ and concentrated under reduced pressure. The crude product was purified through silica gel column chromatography using 50% EtOAc in petroleum ether to afford B (11.6 g, 55%) as a brown solid. ¹HNMR (400 MHz, DMSO-d₆): δ8.05 (br s, 2H), 7.45-7.38 (m, 2H), 7.26 (t, J=7.8 Hz, 1H), 7.08-7.01 (m, 1H), 5.18 (s, 2H), 3.37 (s, 3H).

### Example 3—Synthesis of Intermediate C

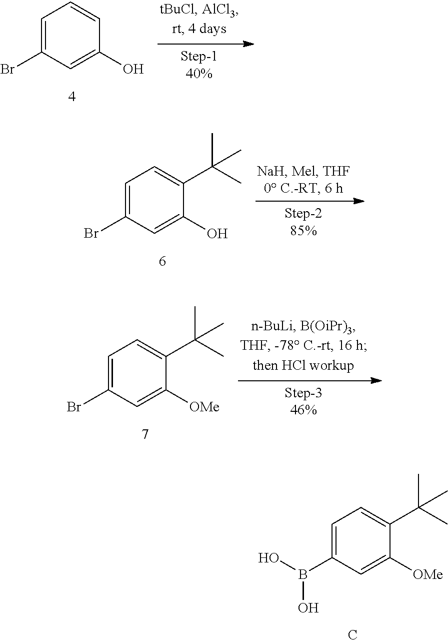

### Synthesis of 5-bromo-2-(tert-butyl)phenol (6)

To a stirred solution of 3-bromophenol (4) (20 g, 115.6 mmol) in tBuCl (200 mL), AlCl₃ (30.83 g, 231.2 mmol) was added and maintained at room temperature for 4 days. The reaction was monitored by TLC. The reaction mixture was diluted with ice-cold water (200 mL) and extracted with EtOAc (2×200 mL). The combined organic layers were dried over anhydrous Na₂SO₄ and concentrated under reduced pressure. The crude product was purified through silica gel column chromatography using 1-2% EtOAc in petroleum ether to afford 6 (8.0 g, 30%) as a colorless liquid. ¹HNMR (400 MHz, CDCl₃): δ7.11 (d, J=8.3 Hz, 1H), 7.06-6.96 (m, 1H), 6.83 (d, J=2.0 Hz, 1H), 4.81 (s, 1H), 1.38 (s, 9H); LCMS (ESI): m/z 226.8 [M−H⁺]; 70.6%; RT=3.57 min (Acquity BEH C18 column, 0.05% formic acid/MeCN with 0.05% formic acid).

### Synthesis of 4-bromo-1-(tert-butyl)-2-methoxybenzene (7)

To a stirred solution of NaH (1.75 g, 43.75 mmol; 60% in mineral oil) in THE (40 mL), was added a solution of 6 (5.0 g, 21.82 mmol) in THE (35 mL) at 0° C. under inert atmosphere. To this, methyl iodide (2.72 mL, 43.67 mmol) was added drop wise at 0° C. and stirred at room temperature for 6 h. After completion of reaction (monitored by TLC), the reaction mixture was quenched with ice-cold water (100 mL) and extracted with EtOAc (2×100 mL). The combined organic layer was dried over anhydrous Na₂SO₄ and concentrated under reduced pressure to afford 7 (4.5 g 85%) as a brown liquid. ¹HNMR (400 MHz, CDCl₃): δ7.12 (d, J=8.3 Hz, 1H), 7.03-6.99 (m, 1H), 6.97 (d, J=2.0 Hz, 1H), 3.83 (s, 3H), 1.34 (s, 9H); GCMS (ESI): m/z 222.1 [M⁺]; 80.9%; RT=8.85 min (ZB-5MS column, 100° C./1 min, 20*C/min/310*C/5.5 min.

### Synthesis of 4-tert-butyl-3-methoxyphenylboronic acid (C)

To a stirred solution of 7 (4.5 g, 18.5 mmol) in THE (90 mL), was added n-butyllithium (12.7 mL, 20.45 mmol; 1.6M solution in hexane) drop wise at −78° C. and stirred at same temperature for 1 h. To this, triisopropyl borate (4.19 g, 22.3 mmol) was added drop wise at −78° C. The reaction was allowed to stir at room temperature for 16 h. After completion of reaction (monitored by TLC), the reaction was quenched with 5N aqueous HCl solution (150 mL) and stirred for 1 h. The reaction solution was extracted with EtOAc (2×150 mL). The combined organic layers were dried over anhydrous Na₂SO₄ and concentrated under reduced pressure. The crude product was purified through silica gel column chromatography using 25% EtOAc in hexane as eluent to afford C (1.8 g, 46%) as a brown solid. ¹HNMR (400 MHz, CDCl₃): δ7.77 (d, J=7.6 Hz, 1H), 7.70 (s, 1H), 7.44 (d, J=7.6 Hz, 1H), 3.97 (s, 3H), 1.43 (s, 9H); LCMS (ESI): m/z 207.1 [M−H⁺]; 88.9%; RT=2.37 min (Acquity BEH C18 column, 0.1% formic acid/MeCN with 0.1% formic acid).

### Example 4—Synthesis of 4-amino-2-(4-(tert-butyl)-3-hydroxyphenyl)-6-(4-fluorophenyl)-5-(piperidin-1-yl)pyridazin-3(2H)-one (11)

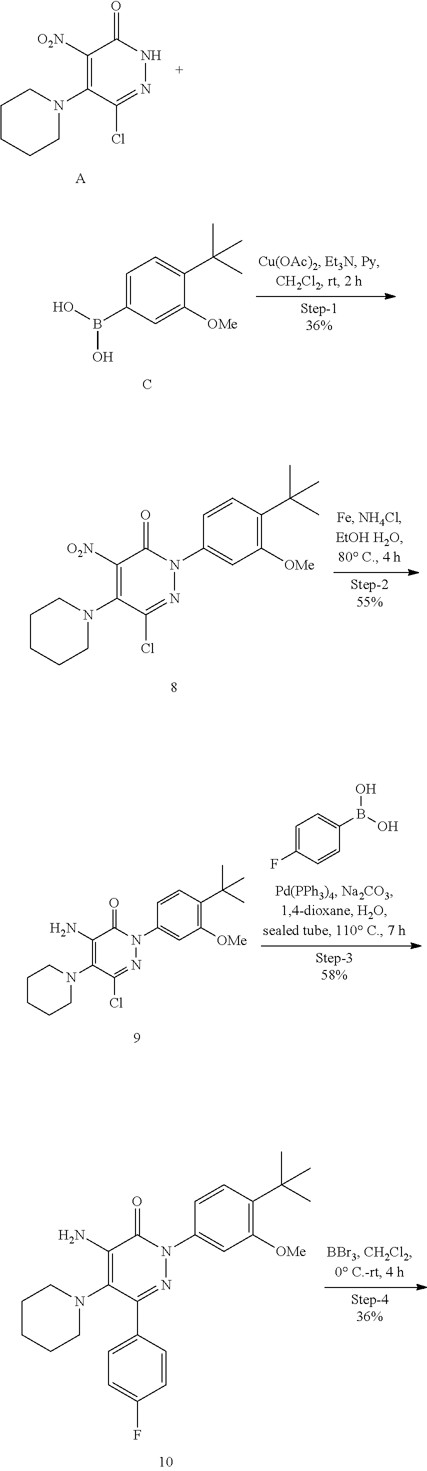

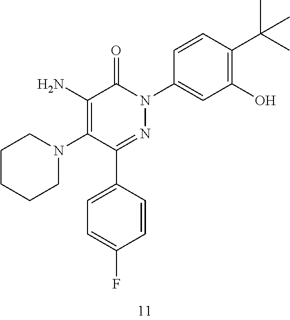 [SMILES: CC(C)(C)c1ccc(-n2nc(-c3ccc(F)cc3)c(N3CCCCC3)c(N)c2=O)cc1O]

### Synthesis of 2-(4-(tert-butyl)-3-methoxyphenyl)-6-chloro-4-nitro-5-(piperidin-1-yl)pyridazin-3(2H)-one (8)

To a stirred solution of A (1.0 g, 3.87 mmol) in CH₂Cl₂ (30 mL), were added C (1.6 g, 7.69 mmol), Cu(OAc)₂ (1.4 g, 7.70 mmol), triethylamine (782 mg, 7.74 mmol) and pyridine (613 mg, 7.75 mmol) sequentially at room temperature and stirred for 2 h. After completion of reaction (monitored by TLC), the reaction mixture was quenched with 2N aqueous HCl solution (100 mL) and extracted with CH₂Cl₂ (2×150 mL). The combined organic layers were washed with water (2×200 mL), dried over anhydrous Na₂SO₄ and concentrated under reduced pressure. The crude product was purified through silica gel column chromatography using 10% EtOAc in hexane as eluent to afford 8 (600 mg, 36%) as a yellow solid. ¹HNMR (400 MHz, CDCl₃): δ 7.33 (d, J=8.3 Hz, 1H), 7.10 (d, J=2.0 Hz, 1H), 7.09-7.04 (m, 1H), 3.85 (s, 3H), 3.32-3.17 (m, 4H), 1.81-1.66 (m, 6H), 1.37 (s, 9H); LCMS (ESI): m/z 421.2 [M+H⁺]; 96.4%; RT=3.19 min (Acquity BEH C18 column, 0.05% formic acid/MeCN with 0.05% formic acid).

### Synthesis of 4-amino-2-(4-(tert-butyl)-3-methoxyphenyl)-6-chloro-5-(piperidin-1-yl)pyridazin-3(2H)-one (9)

To a stirred solution of 8 (600 mg, 1.42 mmol) in ethanol (18 mL), were added Fe powder (239.3 mg, 4.28 mmol), NH₄Cl (457.7 mg, 8.57 mmol) and H₂O (6 mL) at room temperature. The reaction was heated to 80° C. and stirred for 4 h at same temperature. The reaction mixture was cooled to RT, filtered through a short pad of celite. The filtrate was concentrated under vacuum and diluted with EtOAc (200 mL). The resultant solution was washed with water (300 mL), dried over anhydrous Na₂SO₄ and concentrated under reduced pressure. The crude product was purified through silica gel column chromatography using 10% EtOAc in hexane to afford 9 (310 mg, 55%) as a brown solid. ¹HNMR (400 MHz, CDCl₃): δ7.33 (d, J=8.3 Hz, 1H), 7.13-7.04 (m, 2H), 5.42 (br s, 2H), 3.85 (s, 3H), 3.31-3.27 (m, 2H), 2.91-2.96 (m, 2H), 1.77-1.73 (m, 6H), 1.37 (s, 9H); LCMS (ESI): m/z 391.0 [M+H⁺]; 96.7%; RT=4.16 min (Acquity BEH C18 column, 0.05% formic acid/MeCN with 0.05% formic acid).

### Synthesis of 4-amino-2-(4-(tert-butyl)-3-methoxyphenyl)-6-(4-fluorophenyl)-5-(piperidin-1-yl)pyridazin-3(2H)-one (10)

A reaction tube was charged with 9 (300 mg, 0.76 mmol), 1,4-dioxane (15 mL), Na₂CO₃ (163 mg, 1.53 mmol), 4-fluorophenyl boronic acid (215 mg, 1.53 mmol) and purged with argon for 15 min. To this, Pd(PPh₃)₄ (88.8 mg, 0.076 mmol) and H₂O (9 mL) were added and tube was capped. The reaction was heated to 110° C. and stirred for 7 h. After completion of reaction (monitored by TLC), the reaction mixture was cooled to room temperature, diluted with water (100 mL) and extracted with EtOAc (2×100 mL). The combined organic layer was dried over anhydrous Na₂SO₄ and concentrated under reduced pressure. The crude product was purified through silica gel column chromatography using 20% EtOAc in hexane to afford 10 (200 mg, 58%) as a brown solid. ¹HNMR (400 MHz, CDCl₃): δ7.43 (dd, J=5.4, 8.6 Hz, 2H), 7.31 (d, J=8.3 Hz, 1H), 7.16-7.05 (m, 4H), 5.06 (br s, 2H), 3.83 (s, 3H), 2.71-2.68 (m, 4H), 1.56-1.52 (m, 6H), 1.36 (s, 9H); LCMS (ESI): m/z 451.0 [M+H⁺]; 84.1%; RT=4.13 min (Acquity BEH C18 column, 0.05% formic acid/MeCN with 0.05% formic acid).

### Synthesis of 4-amino-2-(4-(tert-butyl)-3-hydroxyphenyl)-6-(4-fluorophenyl)-5-(piperidin-1-yl)pyridazin-3(2H)-one (11)

To a stirred solution of 10 (200 mg, 0.44 mmol) in CH₂Cl₂ (20 mL), was added BBr₃ (0.23 mL, 2.22 mmol) drop wise at 0° C. under inert atmosphere. The reaction was allowed to room temperature and stirred for 4 h. After completion of reaction (monitored by TLC), the reaction mixture was basified with saturated NaHCO₃ solution (10 mL; up to pH˜8) and extracted with CH₂Cl₂ (2×50 mL). The combined organic layer was dried over anhydrous Na₂SO₄ and concentrated under reduced pressure. The crude product was purified by preparative HPLC (Kromasil C 18 column; 5 mM ammonium bicarbonate solution in MeCN) to afford 10 (71 mg, 36%) as a pale yellow solid. ¹HNMR (400 MHz, DMSO-d₆): δ 9.57 (s, 1H; D₂O exchangeable), 7.58-7.44 (m, 2H), 7.25 (t, J=8.9 Hz, 2H), 7.18 (d, J=8.3 Hz, 1H), 7.02 (d, J=2.2 Hz, 1H), 6.96 (dd, J=2.2, 8.3 Hz, 1H), 5.81 (s, 2H; D₂O exchangeable), 2.73-2.68 (m, 4H), 1.48-1.28 (m, 15H); LCMS (ESI): m/z 437.2 [M+H⁺]; 98.9%; RT=2.94 min (Acquity BEH C18 column, 0.05% formic acid/MeCN with 0.05% formic acid); FT-IR (KBr): 3477 (OH), 3369 (1°NH₂), 1604 (C═O) cm⁻¹. CFBE potentiator assay pot. pEC₅₀=8.0.

### Example 5—Synthesis of 4-amino-2-(3-hydroxyphenyl)-5-(piperidin-1-yl)-6-(o-tolyl)pyridazin-3(2H)-one (15)

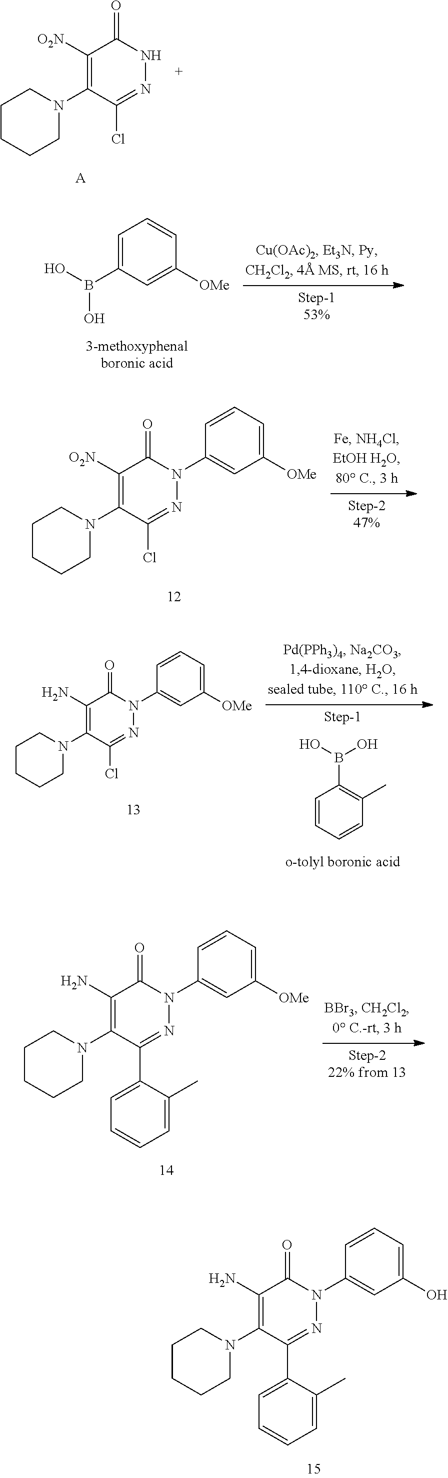

### Synthesis of 6-chloro-2-(3-methoxyphenyl)-4-nitro-5-(piperidin-1-yl)pyridazin-3(2H)-one (12)

To a stirred solution of A (800 mg, 3.09 mmol) and 3-methoxyphenylboronic acid (700 mg g, 4.64 mmol) in CH₂Cl₂ (20 mL), were added Cu(OAc)₂ (840 mg, 4.64 mmol), triethylamine (0.86 mL, 6.18 mmol), pyridine (0.5 mL, 6.18 mmol) and 4 Å molecular sieves (150 mg) sequentially at room temperature and stirred for 16 h. After completion of reaction (monitored by TLC), the reaction mixture was filtered through a celite pad and washed with CH₂Cl₂ (10 mL). The filtrate was washed with water (50 mL) and the aqueous layer was extracted with CH₂Cl₂ (2×50 mL). The combined organic layers were dried over anhydrous Na₂SO₄ and concentrated under reduced pressure. The crude product was purified through silica gel column chromatography using 25% EtOAc in petroleum ether as eluent to afford 11 (600 mg, 53%) as a yellow solid. ¹HNMR (400 MHz, CDCl₃): δ7.36 (t, J=8.3 Hz, 1H), 7.19-7.13 (m, 2H), 6.93-6.89 (m, 1H), 3.81 (s, 3H), 3.24-3.21 (m, 4H), 1.78-1.68 (m, 6H); LCMS (ESI): m/z 365.1 [M+H⁺]; 95.1%; RT=8.39 min (XSelect CSH C18 column, 5 mM aqueous ammonium acetate with MeCN).

### Synthesis of 4-amino-6-chloro-2-(3-methoxyphenyl)-5-(piperidin-1-yl)pyridazin-3(2H)-one (13)

To a stirred solution of 12 (650 mg, 1.78 mmol) in ethanol (13 mL), were added Fe powder (300 mg, 5.34 mmol), NH₄Cl (570 mg, 10.68 mmol) and H₂O (6.5 mL) at room temperature. The reaction was heated to 80° C. and stirred for 3 h. The reaction mixture was cooled to room temperature and filtered through a short pad of celite. The filtrate was concentrated under reduced pressure. The residue obtained was diluted with water (20 mL) and extracted with CH₂Cl₂ (2×25 mL). The combined organic layer was dried over anhydrous Na₂SO₄ and concentrated under reduced pressure. The crude product was purified through silica gel column chromatography using 20% EtOAc in petroleum ether to afford 12 (280 mg, 47%) as a brown solid. ¹HNMR (400 MHz, CDCl₃): δ 7.41-7.29 (m, 1H), 7.18 (d, J=8.1 Hz, 1H), 7.14 (d, J=2.0 Hz, 1H), 6.92 (dd, J=2.0, 8.1 Hz, 1H), 5.43 (br s, 2H), 3.83 (s, 3H), 3.29-3.26 (m, 2H), 2.92-2.86 (m, 2H), 1.76-1.70 (m, 6H); LCMS (ESI): m/z 335.1 [M+H⁺]; 92.8%; RT=3.67 min (Acquity BEH C18 column, 0.05% formic acid/MeCN with 0.05% formic acid).

### Synthesis of 4-amino-2-(3-methoxyphenyl)-5-(piperidin-1-yl)-6-(o-tolyl)pyridazin-3(2H)-one (14)

A reaction tube was charged with 13 (400 mg, 1.19 mmol), o-tolylboronic acid (490 mg, 3.58 mmol), Na₂CO₃ (380 mg, 3.58 mmol), 1,4-dioxane (8 mL), H₂O (0.8 mL) and degassed by purging with argon for 10 min. To this, Pd(PPh₃)₄ (83 mg, 0.071 mmol) was added and purged again with argon for 15 min. The reaction tube was capped and stirred at 110° C. for 16 h. The reaction mixture was cooled to room temperature and concentrated under reduced pressure. The residue was diluted with water (25 mL) and extracted with EtOAc (2×25 mL). The combined organic layer was dried over anhydrous Na₂SO₄ and concentrated under reduced pressure. The crude product was purified through silica gel column chromatography using 30% EtOAc in petroleum ether to afford 14 (280 mg; 65% pure) as a brown semi solid, which was directly taken for next reaction without further purification. ¹HNMR (400 MHz, CDCl₃): δ 7.49-7.40 (m, 1H), 7.37-7.29 (m, 3H), 7.20-7.16 (m, 3H), 6.88 (dd, J=1.8, 8.3 Hz, 1H), 5.08 (br s, 2H), 3.81 (s, 3H), 2.30 (s, 3H), 2.62-2.59 (m, 4H), 1.51-1.43 (m, 4H), 1.39-1.35 (m, 2H); LCMS (ESI): m/z 391.2 [M+H⁺]; 65.6%; RT=2.79 min (Acquity BEH C18 column, 0.05% formic acid/MeCN with 0.05% formic acid).

### Synthesis of 4-amino-2-(3-hydroxyphenyl)-5-(piperidin-1-yl)-6-(o-tolyl)pyridazin-3(2H)-one (15)

To a stirred solution of 14 (280 mg) in CH₂Cl₂ (10 mL), was added BBr₃ (3.5 mL, 3.58 mmol; 1M solution in CH₂Cl₂) drop wise at 0° C. under inert atmosphere. The reaction was allowed to room temperature and stirred for 3 h. After completion of reaction (monitored by TLC), the reaction mixture was diluted with water (5 mL), basified with saturated NaHCO₃ solution (10 mL; up to pH˜8) and extracted with CH₂Cl₂ (2×15 mL). The combined organic layer was dried over anhydrous Na₂SO₄ and concentrated under reduced pressure. The crude product was purified by preparative HPLC (XBridge C 18 column; 10 mM aqueous ammonium bicarbonate solution in MeCN) to afford 15 (98 mg, 22% from 12) as a white solid. ¹HNMR (400 MHz, DMSO-d₆): δ 9.62 (br s, 1H; D₂O exchangeable), 7.37-7.26 (m, 2H), 7.26-7.19 (m, 3H), 7.04-6.93 (m, 2H), 6.74 (ddd, J=1.0, 2.4, 8.2 Hz, 1H), 5.78 (br s, 2H; D₂O exchangeable), 2.63-2.58 (m, 4H), 2.21 (s, 3H), 1.40-1.18 (m, 6H); LCMS (ESI): m/z 377.1 [M+H⁺]; 99.1%; RT=2.50 min (Acquity BEH C18 column, 0.05% formic acid/MeCN with 0.05% formic acid); FT-IR (KBr): 3483 (OH), 3368 (1° NH), 1594 (C═O) cm⁻¹. CFBE potentiator assay pot. pEC₅₀=7.8.

### Example 6—Synthesis of 4-amino-5-(dimethylamino)-2-(3-hydroxyphenyl)-6-phenylpyridazin-3(2H)-one (21)

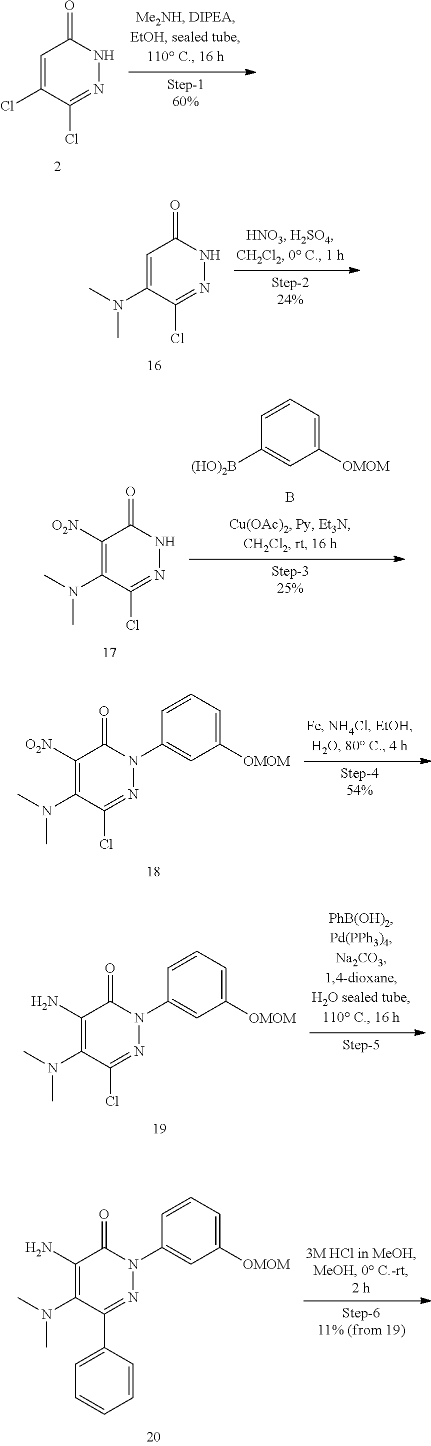

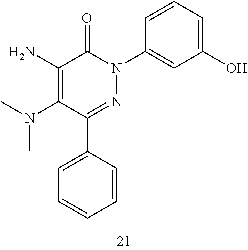 [SMILES: CN(C)c1c(-c2ccccc2)nn(-c2cccc(O)c2)c(=O)c1N]

### Synthesis of 6-chloro-5-(dimethylamino)pyridazin-3(2H)-one (16)

A reaction tube was charged with 2 (5.0 g, 30.5 mmol), dimethyl amine (30.4 mL, 2M solution in THF, 61.0 mmol), diisopropylethylamine (26.5 mL, 152.5 mmol) and ethanol (60 mL) at RT under inert atmosphere. The reaction tube was capped and stirred at 110° C. for 16 h. After complete consumption of starting material (monitored by TLC), the reaction mixture was cooled to RT and concentrated under reduced pressure. The residue obtained was diluted with water (200 mL) and filtered. The solid was washed with diethyl ether (50 mL) to afford 16 (3.2 g, 60%) as an off-white solid. ¹HNMR (400 MHz, DMSO-d₆): δ 12.59 (br s, 1H), 6.01 (s, 1H), 2.85 (s, 6H); LCMS (ESI): m/z 173.8 [M+H⁺]; 99.8%; RT=2.04 min (Acquity BEH C18 column, 0.05% formic acid/MeCN with 0.05% formic acid).

### Synthesis of 6-chloro-5-(dimethylamino)-4-nitropyridazin-3(2H)-one (17)

To a stirred solution of 16 (1.5 g, 8.64 mmol) in CH₂Cl₂ (25 mL), was added concentrated H₂SO₄ (9 mL) followed by fuming nitric acid (2.4 mL) drop wise at 0° C. and stirred at same temperature for 1 h. Reaction was monitored by TLC. The reaction mixture was diluted with ice-cold water (100 mL) and extracted with CH₂Cl₂ (2×40 mL). The combined organic layer was washed with water (2×50 mL), dried over anhydrous Na₂SO₄ and concentrated under reduced pressure to afford 17 (460 mg, 24%) as a yellow solid. ¹HNMR (400 MHz, CDCl₃): δ 10.80 (br s, 1H), 3.04 (s, 6H); LCMS (ESI): m/z 218.9 [M+H⁺]; 86.2%; RT=2.31 min (Acquity BEH C18 column, 0.05% formic acid/MeCN with 0.05% formic acid).

### Synthesis of 6-chloro-5-(dimethylamino)-2-(3-(methoxymethoxy)phenyl)-4-nitropyridazin-3(2H)-one (18)

To a stirred solution of 17 (2.0 g, 9.15 mmol) in CH₂Cl₂ (40 mL), were added (3-(methoxymethoxy)phenyl)boronic acid (3.33 g, 18.3 mmol), Cu(OAc)₂ (3.33 g, 18.3 mmol), triethylamine (2.6 mL, 18.3 mmol), pyridine (1.5 mL, 18.3 mmol) sequentially at room temperature and stirred for 16 h. After completion of reaction (monitored by TLC), the reaction mixture was filtered through a Celite pad and the filtrate was concentrated under reduced pressure. The residue was diluted with water (100 mL) and extracted with EtOAc (50 mL). The EtOAc layer was dried over anhydrous Na₂SO₄ and concentrated under reduced pressure. The obtained crude product was purified through silica gel column chromatography using 10-15% EtOAc gradient in petroleum ether to afford 18 (800 mg, 25%) as a yellow solid. ¹HNMR (400 MHz, DMSO-d₆): δ 7.43 (t, J=8.1 Hz, 1H), 7.23-7.09 (m, 3H), 5.22 (s, 2H), 3.39 (s, 3H), 2.99 (s, 6H); LCMS (ESI): m/z 355.15 [M+H⁺]; 84.9%; RT=2.33 min (Acquity BEH C18 column, 0.05% formic acid/MeCN with 0.05% formic acid).

### Synthesis of 4-amino-6-chloro-5-(dimethylamino)-2-(3-(methoxymethoxy)phenyl)pyridazin-3(2H)-one (19)

To a stirred solution of 18 (800 mg, 2.25 mmol) in ethanol (16 mL), were added Fe-powder (380 mg, 6.75 mmol), NH₄Cl (720 mg, 13.5 mmol) and H₂O (8 mL) at room temperature. The reaction was heated to 80° C. and stirred for 4 h. The reaction mixture was cooled to room temperature and filtered through a short pad of celite. The filtrate was concentrated under reduced pressure. The residue obtained was diluted with water (50 mL) and extracted with EtOAc (2×30 mL). The combined organic layer was dried over anhydrous Na₂SO₄ and concentrated under reduced pressure to afford 19 (400 mg, 54%) as a brown solid. ¹HNMR (400 MHz, DMSO-d₆): δ 7.35-7.49 (m, 1H), 7.13-7.23 (m, 2H), 7.03-7.12 (m, 1H), 6.63 (br s, 2H), 5.22 (s, 2H), 3.39 (s, 3H), 2.73 (s, 6H); LCMS (ESI): m/z 325.1 [M+H⁺]; 93.6%; RT=2.31 min (Acquity BEH C18 column, 0.05% formic acid/MeCN with 0.05% formic acid).

### Synthesis of 4-amino-5-(dimethylamino)-2-(3-(methoxymethoxy)phenyl)-6-phenylpyridazin-3(2H)-one (20)

A reaction tube was charged with 19 (400 mg, 1.23 mmol), 1,4-dioxane (8 mL), Na₂CO₃ (260 mg, 2.46 mmol), benzeneboronic acid (300 mg, 2.46 mmol), Pd(PPh₃)₄ (142 mg, 0.123 mmol) and H₂O (4 mL) and purged with argon for 10 min. The tube was capped and stirred at 110° C. for 16 h. After completion of reaction (monitored by TLC), the reaction mixture was cooled to room temperature and filtered through a celite pad. The filtrate was concentrated under reduced pressure. The residue obtained was diluted with water (50 mL) and extracted with EtOAc (2×20 mL). The combined organic layer was dried over anhydrous Na₂SO₄ and concentrated under reduced pressure. The crude product was purified through silica gel column chromatography using 50% EtOAc in petroleum ether to afford 300 mg of 20 (purity ˜45%; brown solid) as a mixture with triphenylphosphene oxide (35%), which was directly taken for next step without further purification. LCMS (ESI): m/z 367.1 [M+H⁺]; 45.4%; RT=3.07 min (Acquity BEH C18 column, 0.05% formic acid/MeCN with 0.05% formic acid).

### Synthesis of 4-amino-5-(dimethylamino)-2-(3-hydroxyphenyl)-6-phenylpyridazin-3(2H)-one (21)

To a stirred solution of 20 (300 mg; purity ˜45%) in methanol (3 mL), was added 3M HCl solution in methanol (3 mL) drop wise at 0° C. under inert atmosphere. The reaction was allowed to stir at room temperature for 2 h. After completion of reaction (monitored by TLC), methanol was concentrated. The obtained residue was diluted water (20 mL) and extracted with EtOAc (2×10 mL). The combined organic layer was dried over anhydrous Na₂SO₄ and concentrated under reduced pressure. The crude product was purified by preparative HPLC (Kinetex phenylhexyl C 18 column; 5 mM aqueous ammonium bicarbonate solution in MeCN) to afford 21 (85 mg; 11% from 5 in two steps) as an brown solid. ¹HNMR (400 MHz, DMSO-d₆): δ9.61 (br s, 1H, D₂O exchangeable), 7.53-7.38 (m, 5H), 7.29-7.17 (m, 1H), 7.07-6.96 (m, 2H), 6.76 (ddd, J=8.1, 2.4, 1.0 Hz, 1H), 5.98 (br s, 2H; D₂O exchangeable), 2.46 (s, 6H); LCMS (ESI): m/z 323.1 [M+H⁺]; 99.8%; RT=2.05 min (Acquity BEH C18 column, 0.05% formic acid/MeCN with 0.05% formic acid). CFBE potentiator assay pot. pEC₅₀=7.7.

### Compound 11—In-Vitro Model of Chronic Obstructive Pulmonary Disorder

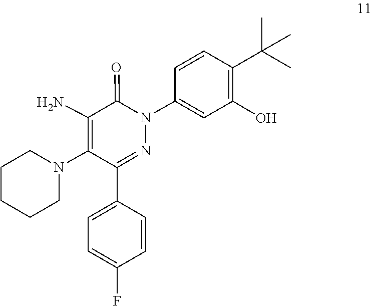 [SMILES: CC(C)(C)c1ccc(-n2nc(-c3ccc(F)cc3)c(N3CCCCC3)c(N)c2=O)cc1O]

The CFTR chloride channel is situated on the luminal membrane of airway surface epithelium and serves to enhance the fluidity of the airway surface liquid, thereby preventing mucus obstruction. Tobacco smoke reduces the functional expression of CFTR on the epithelial surface, exacerbating mucostasis and obstruction (A. Rab, S. M. Rowe, S. V. Raju, Z. Bebok, S. Matalon and J. F. Collawn. Am J Physiol Lung Cell Mol Physiol 2013 Vol. 305 Issue 8 Pages L530-41). These findings support the hypothesis that smoke exposure induces CFTR deficiency, also known as “acquired Cystic Fibrosis” and small molecules that potentiate activity of the remaining CFTR function, will reverse the initial stages of COPD pathogenesis. Ivacaftor, an approved potentiator of CFTR was shown to reverse mucostasis in certain tissue culture models and a novel CFTR potentiator called Icenticaftor has shown early promise in restoring lung function in clinical trials of people with COPD (S. M. Rowe, I. Jones, M. T. Dransfield, N. Haque, S. Gleason, K. A. Hayes, et al. Int J Chron Obstruct Pulmon Dis 2020 Vol. 15 Pages 2399-2409).

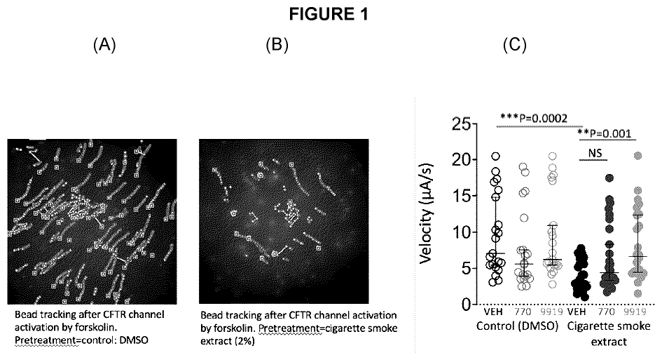

Compound 11 was tested in an in-vitro model of COPD. In this model, primary differentiated human bronchial epithelial cultures were exposed to cigarette smoke extract (4,5). Exposure to cigarette smoke extract (CSE) led to mucostasis and slowed mucociliary movement (FIG. 1(A) and (B)). We found that Compound 11 prevented the mucostasis induced by cigarette smoke extract when added to differentiated airway epithelial cultures (FIG. 1(C)). Changes in mucociliary movement were determined as in our previous publication (7), i.e., by tracking the speed of fluorescent beads added to the surface of the airway cultures. The relative rescue effect of Compound 11 compared favourably relative to the competitor, the FDA approved CFTR potentiator marketed as Ivacaftor (VX-770).

FIG. 1 shows that the compound enhances mucociliary movement on the surface of primary bronchial cultures previously exposed to cigarette smoke extract. The images on the left ((A) and (B)) show the negative impact of cigarette smoke extract (CSE) on fluorescent bead tracking reflecting the development of sticky mucus on the surface liquid on primary bronchial cultures. In the graph on the right (C), it shows the quantification of bead velocity on the surface of primary bronchial airway cultures under control conditions (cultures from 4 donors with vehicle alone, DMSO, open circles). Ivacaftor (VX-770 circles) or Compound 11 circles) were added at the time of the vehicle for 24 hours. Neither VX-770 nor Compound 11 had any impact on bead velocity under control conditions. Treatment with CSE (2%/volume, solid black symbols) caused a significant decrease in bead velocity in cultures from 5 donors. The velocity of beads on bronchial cultures exposed to CSE, treated with Compound 11 potentiator (solid symbols) was rescued to control values. Each symbol represents the mean bead velocity in 5 videos taken at randomly selected regions on each airway culture.

### a Novel Potentiator of Wt-CFTR Channel Activity.

### Materials and Methods

Cell-attached patch clamp studies of CFTR channel potentiation by novel potentiator compound.

HEK-293 cells stably expressing wild-type human CFTR in patch clamp studies of single channel activity were used. Cells were the generous gifts of D. Rotin (SickKids Hospital, Toronto). CFTR Cl⁻ channels were recorded in cell-attached membrane patches using an Axopatch 200A patch clamp amplifier and pCLAMP software (both from Molecular Devices, Sunnyvale, CA). The pipette and bath (extracellular) solutions contained 140 mm N-methyl-d-glucamine, 140 mm aspartic acid, 5 mm CaCl₂, 2 mm MgSO₄, and 10 mm TES, adjusted to pH 7.3 with Tris ([Cl⁻]: 10 mM). The bath was maintained at room temperature.

In order to activate Wt-CFTR channels, 10 uM forskolin was added to the bath. CFTR mediated channel openings were inhibited by application of CFTRInh-172 to the bath. CFTR Cl⁻ channels were potentiated by the addition of SK-POT (compound 11) (10 μM) to the bath. To determine channel number, we used the maximum number of simultaneous channel openings observed during the experimental procedure. Single channel recordings were filtered and digitized data as described previously. To measure single-channel current amplitude, Gaussian distributions were fitted to current amplitude histograms. To measure Pₒ, lists of open and closed times were created using a half-amplitude crossing criterion for event detection, and dwell time histograms were constructed and fitted. For the purpose of illustration, single-channel records were filtered at 500 Hz.

### Calu-3 CSE Experiments

Calu-3 cells were cultured using EMEM (Wisent) supplemented with FBS at 20% (v/v) and 1× Penicillin/Streptomycin (Wisent). Calu-3 cells were seeded on clear-bottom, black-walled 96 well plates (Costar, Corning) at a density of 10,000 cells/well and cultured for 2 days post-confluence. Cells then received the chronic treatment of toxin and potentiator. The toxin treatment consisted of either cigarette smoke extract (CSE) prepared in 100% DMSO (University of Alabama at Birmingham), dissolved at a concentration of 2% (v/v) in Calu-3 medium, with an equivalent concentration of DMSO alone serving as the toxin control. The chronic drug treatment was co-applied with the toxin, which included either VX-770 or Compound 11 (both prepared in 100% DMSO) dissolved in Calu-3 medium at a final concentration of 1 μm. VX-770 (Vertex) and Compound 11 solutions were prepared from 1 mM stocks. The control for the drug treatment was the vehicle, DMSO at an equivalent volume. Combining the two toxin treatments and three drug treatments, a total of six treatments were applied chronically to 4 wells per condition, for a total of 24 wells receiving chronic treatment.

Cells were cultured for an additional 24 hours after chronic treatment application, after which cells were incubated with FLiPR assay buffer. The FLiPR assay buffer consisted of blue FLiPR dye (Molecular Devices) dissolved at a concentration of 0.5 mg/mL in chloride-free buffer (150 mM NMDG, 150 mM gluconolactone, 3 mM potassium gluconate, 300 mOsm and pH 7.38). Buffer was incubated with cells at 37 degrees and 5% CO₂ for 35 minutes. CFTR function was assessed by measuring changes in fluorescence activity after acute CFTR channel activation by addition of cAMP agonist forskolin dissolved in chloride-free buffer to a final concentration 1 μm, using the SpectraMax i3x multimodal plate reader. Fluorescence readings (excitation 530 nm; emission 560 nm) for each well were taken at 30-second intervals for 5 minutes (baseline) or 10 minutes (activation). CFTR activity was then terminated by addition of CFTR-inhibitor 172 dissolved in chloride-free buffer to a concentration of 10 μm, to further verify the specificity of the response to CFTR activity. Fluorescence changes were recorded at 30-second intervals for another 10 minutes. In analysis, all fluorescence values were normalized per well to the final reading prior to the addition of forskolin and expressed as a percentage of this reading.

### Bead Tracking Assay of Mucociliary Movement

Mature bronchial epithelial cultures, seeded on 24-well transwell inserts, derived from non-smoker donors of variable ages were received from the University of Iowa and cultured at air-liquid interface (ALI) using UltraG medium. A total of 6 inserts per donor were cultured, and all inserts from a single donor were selected to receive chronic drug and toxin treatment. Ahead of chronic treatment, cultures were subject to mucomist to reduce the volume and viscosity of the mucous layer ahead of imaging. Cultures were first washed twice with 200 uL HBSS (Wisent) applied apically to the transwell, each wash for 10 minutes at 37 degrees and 5% CO₂. Cells were then incubated with 10 μM N-acetyl-cysteine (source?) dissolved in HBSS (Wisent) for 30 minutes, followed by a final HBSS wash as described previously. The toxin treatment consisted of either cigarette smoke extract (CSE) prepared in 100% DMSO (University of Alabama at Birmingham), dissolved at a concentration of 2% (v/v) in UltraG medium, with an equivalent concentration of DMSO alone serving as a control. The chronic drug treatment was co-applied with the toxin treatment, which included either VX-770 (Vertex) or SK-POT, aka Compound 11 (both prepared in 100% DMSO) dissolved in UltraG medium at a final concentration of 1 μm. There were a total of 6 chronic treatments, with each of the 6 inserts per donor receiving a different chronic treatment. Bronchial epithelia were incubated with the chronic treatment, applied apically, for 24 hours at 37 degrees C. and 5% CO₂. After 24 hours, green fluorescent microspheres (brand?) dissolved in HBSS at a concentration of 0.05% (v/v) were applied apically; cultures were incubated with the microspheres for 45 minutes. The toxin and potentiator treatments were carried forth into the microsphere solution.

Inserts were then imaged at room temperature by epifluorescence microscopy (Zeiss or Olympus). The basic replicate of the assay consists of a 5-second video captured in the green channel and 40-60 frames per second; Z-position was adjusted ahead of each video to ensure beads in the airway surface liquid stratum immediately above the cilia were being captured. 5 videos were captured per insert/conditions, representing the centre and 4 corners of the insert. 2 sets of 5 videos were captured per insert/condition: one at baseline, and one 20 minutes following CFTR stimulation by forskolin at a concentration of 10 μM. Inserts were incubated at room temperature after forskolin addition.

Videos were captured using Volocity software and exported as one TIF file per frame. Frames were then re-compiled into complete videos using Arivis 4D software. Using an algorithm as previously described in Wu et al. 2017, microspheres were highlighted by adjusting two parameters: fluorescence intensity and diameter. Parameters were adjusted until the software only identified singular beads as such; the success of identifying singular beads was manually verified at the beginning, middle, and end of each video. After highlighting the beads, the algorithm generated a displacement value for each bead, along with a value for the number of frames the algorithm tracked the bead. Beads tracked for less than 5 frames were excluded from analysis. Velocity (in μm/s) was then calculated (per bead analyzed) by dividing generated velocity by the number of frames tracked, and then multiplying by the frames per second. Velocity values were compiled, and a mean velocity calculated for each video.

### Calu-3 Western Blots

Calu3 and were lysed in radioimmunoprecipitation (RIPA) buffer (50 mM Tris-HCl, 1 mM EDTA, 150 mM NaCl, pH 7.4) with 0.1% SDS, 0.1% Triton X-100, and a protease inhibitor cocktail (Roche) (1×). The lysis was performed for 5 minutes, on ice, using 40 μL of RIPA buffer as described. Lysed cells were collected from individual wells by means of scraping with a micropipette tip, and subsequently centrifuged at 4 degrees Celsius and 15,000 rpm for 10 minutes. Lysates were then analyzed by SDS-PAGE using 6% Tris-Glycine gels (Invitrogen); transfer was performed to nitrocellulose membranes (Bio-Rad), at 100 mV and 1 hour. Following blocking with 5% (w/v) skim milk dissolved in PBS-Tween, CFTR was probed overnight, at 4 degrees Celsius, with primary antibody CFTR-NBD2-specific murine mAb 596 dissolved in blocking buffer at a 1:2000 dilution. The loading control, calnexin, was probed with rat anti-calnexin dissolved in blocking buffer at a 1:10000 dilution. Horseradish peroxidase (HRP) tagged anti-mouse and anti-rat antibodies, both diluted in blocking buffer at 1:5000, were employed as secondary antibodies in a 1-hour room temperature incubation. Blots were developed using ECL reagent (Bio-Rad) and imaged using the Li-Cor Odyssey Fc (LI-COR Biosciences) with a 2 minute exposure. Relative CFTR levels were quantified using ImageStudioLite (LI-COR Biosciences).

### Results and Discussion

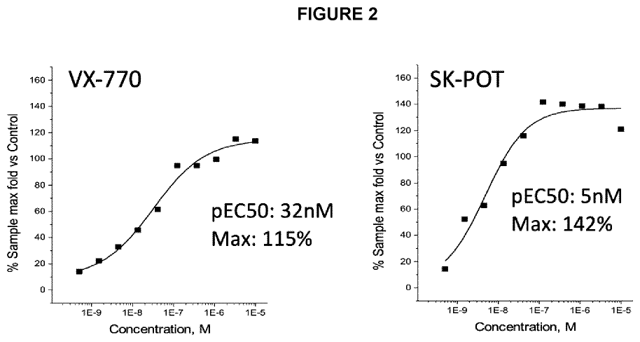

FIG. 2, shows the dose response of SK-POT (compound 11) activity relative to VX-770, the highly effective potentiator compound used in the treatment of Cystic Fibrosis. In these studies, F508del-CFTR was endogenously expressed in CFBE41o⁻ cells and it's trafficking defect corrected by low temperature (27 degrees Celsius) incubation. The fold increase in cyclic AMP dependent F508del-CFTR mediated chloride channel activity induced by potentiator treatment was determined using the fluorescence-based, membrane potential difference assay previously described. As shown in FIG. 1, the potency of Compound 11 was found to be 5 nM relative to 32 nM as determined for VX-770.

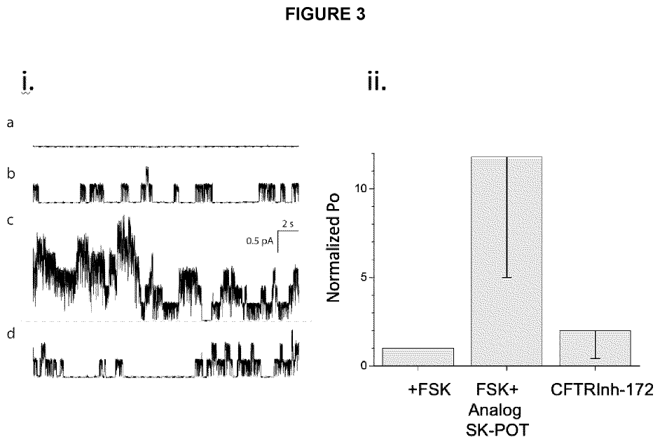

Detailed analysis of the activity of this new class of small molecules in potentiating Wt-CFTR were conducted in patch clamp studies in HEK-293 cells stably expressing this gene. In FIG. 3(i), traces are shown obtained in the cell-attached mode, where the cells were sequentially exposed to forskolin, then the structural analog of compound 11, followed by the CFTR channel inhibitor, CFTRInh172 (10 uM). While only one active channel was apparent in the presence of forskolin, a total of 8 channels were active in the presence of the potentiator compound. After the addition of CFTRinh-172, there was a significant decrease in the number of active channels. The bar graph in FIG. 3(ii), shows the CFTR channel activity after normalization for the total number of channels (n=8). There was clearly an increase in channel open probability with the addition of the novel compound as expected for a CFTR potentiator.

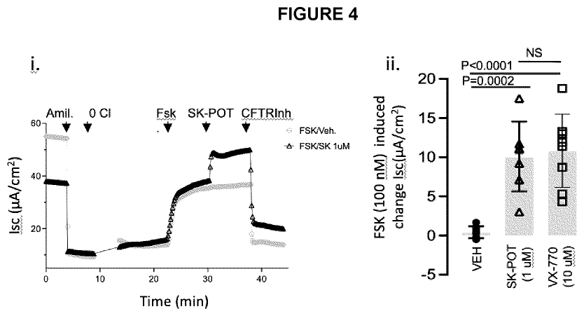

The impact of SK-POT (compound 11) in potentiating Wt-CFTR mediated transepithelial chloride conduction in primary human bronchial epithelial cultures was then tested. As shown in FIG. 4, forskolin activated, short circuit currents mediated by Wt-CFTR in primary bronchial epithelial cultures in Ussing chamber studies, were potentiated by SK-POT (1 μM). Representative traces are shown in the left panel and the scatter plots on the right show that these results are reproducible and statistically significant. Interestingly, the potentiation induced by 1 μM SK-POT was similar to that achieved by ivacaftor (VX-77) at 10 μM.

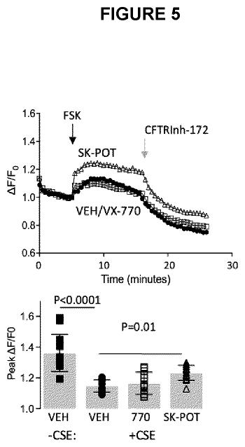

It was then determined if SK-POT compound was capable of ameliorating the negative effect of cigarette smoke (CSE) on Wt-CFTR channel function. The cell line, Calu-3, has been employed extensively in studies of the regulation for Wt-CFTR as this airway epithelial cell line endogenously expresses this channel following its differentiation CFTR channel activity was measured using the FLIPR (fluorescence-based plate reader assay). Firstly, we recapitulated the previously published, detrimental effect of cigarette smoke extract on CFTR channel activity (FIG. 5i, top and bottom panels). In paired studies, (3 biological replicates with 3-4 technical replicates) the effect of VX-770 and SK-POT, both at 1 uM, in potentiating CFTR activity after CSE exposure was compared. As shown in the bar graph of FIG. 5, only (SK-POT) treatment led to a significant increase in forskolin-activated CFTR channel activity.

Mucostasis, a primary defect in COPD has been modeled in-vitro as reduced motility of fluorescent nanoparticles, seeded in the mucus-containing liquid on top of well-differentiated tracheal airway cultures (Y. S. Wu, J. Jiang, S. Ahmadi, A. Lew, O. Laselva, S. Xia, et al. Mol Pharmacol 2019 Vol. 96 Issue 4 Pages 515-525).

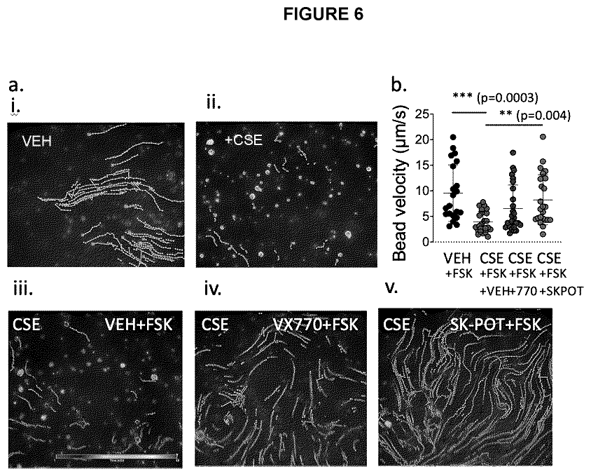

Fluorescent nanoparticles were seeded in the airway surface and tracked bead movement. Bead trajectories, as shown in the images of FIG. 6. The top images (a,i and ii) show that bead displacement is reduced following exposure to CSE as expected. We then assessed the effect of pretreatment with the potentiators, VX-770 (1 μM) or SK-POT (1 μM) on CSE-altered muco-ciliary movement (panels a.iii-v), and in FIG. 6(b), bead velocity against pretreatment with specified potentiators. In 5 biological replicates, 4-5 technical replicates (or videos), pretreatment with SK-POT produced a significant increase in bead velocity in CSE treated, primary, bronchial cultures. Interestingly, in these studies, a significant increase in the forskolin induced response in CSE-treated cultures after VX-770 addition was not observed.

### DISCUSSION

The current studies show that potentiators of CFTR channel activity have the potential to ameliorate mucostasis induced on human bronchial epithelial cells by cigarette smoke extract (CSE). Further, the findings indicate that potentiators that promote superior CFTR channel activity in CSE exposed epithelial cells, such as the potentiator described here, will also be more effective in preventing CSE associated mucostasis. Together, these studies support the future in-vivo evaluation of SK-POT as a therapeutic intervention for COPD associated bronchitis.

It was found that acute treatment with the SK-POT compound was superior to VX-770 at 1 micromolar (approximate EC90 for both) with respect to potentiating CFTR channel activity in epithelial cells that had been exposed to cigarette smoke. The superior effect of the SK-POT compound may reflect a different molecular mechanism of action relative to VX-770. In the present studies, while we did observe a large decrease in CFTR channel function in Calu-3 cells after CSE pre-treatment, a significant decrease in steady state abundance of mature Band C was not observed.

The positive effect of the SK-POT compound in enhancing CFTR channel activity in CSE exposed epithelial cells, translated to efficacy in rescuing defective mucociliary movement in CSE-exposed primary bronchial cultures (FIG. 6). This observation is consistent with the proposal that augmenting CFTR function has the potential to ameliorate mucus aggregation and obstruction in COPD. Interestingly, there was considerable variability in bead velocities (FIG. 6), amongst donor-specific cultures. This variability may reflect donor specific differences in the transport properties of primary bronchial cultures as well as certain heterogeneity within each culture, vis a vis the proportion of ciliated cells.

In summary, these data support that CFTR channel activity in tissue culture models of COPD will prevent the mucus aggregation and mucostasis. While the efficacy of SK-POT in preventing mucostasis on the surface of human bronchial epithelial cultures is variable amongst donor specific cultures—the mean response was substantive, relative to Ivacaftor.

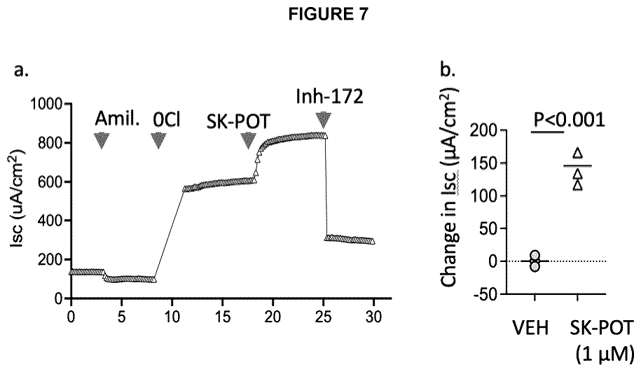

FIG. 7 shows that K-POT compound is also effective in augmenting CFTR channel function in ferret bronchial tissue—the preferred animal model for preclinical studies of interventions targeting airway diseases (N. Kaza, V Y Lin et al. Eur Respir J. 2022 Jul. 13; 60(1):2101581).

TABLE 1
pEC₅₀ values for Compounds of the Disclosure

| | CFBE potentiator | Mass |
|---|---|---|
| Compound | EC₅₀ range | (found) |
| 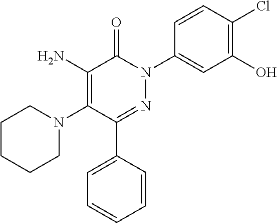 [SMILES: Nc1c(N2CCCCC2)c(-c2ccccc2)nn(-c2ccc(Cl)c(O)c2)c1=O] | 1 nM-100 nM | 397.12 |
| 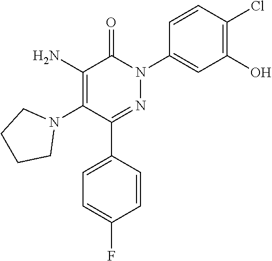 [SMILES: Nc1c(N2CCCC2)c(-c2ccc(F)cc2)nn(-c2ccc(Cl)c(O)c2)c1=O] | 1 nM-100 nM | 401.16 |
| 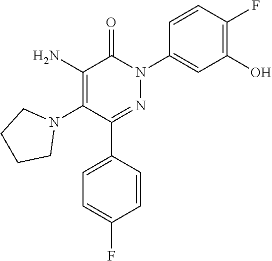 [SMILES: Nc1c(N2CCCC2)c(-c2ccc(F)cc2)nn(-c2ccc(F)c(O)c2)c1=O] | 1 nM-100 nM | 385.18 |
| 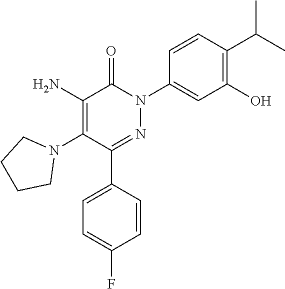 [SMILES: CC(C)c1ccc(-n2nc(-c3ccc(F)cc3)c(N3CCCC3)c(N)c2=O)cc1O] | 1 nM-100 nM | 409.23 |
| 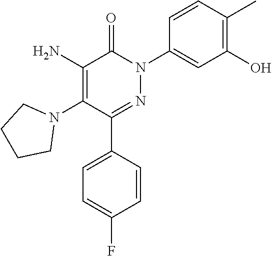 [SMILES: Cc1ccc(-n2nc(-c3ccc(F)cc3)c(N3CCCC3)c(N)c2=O)cc1O] | 1 nM-100 nM | 381.15 |
| 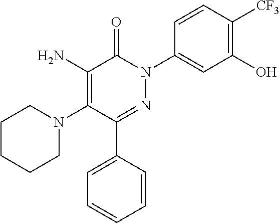 [SMILES: Nc1c(N2CCCCC2)c(-c2ccccc2)nn(-c2ccc(C(F)(F)F)c(O)c2)c1=O] | 1 nM-100 nM | 431.15 |
| 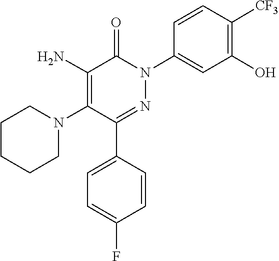 [SMILES: Nc1c(N2CCCCC2)c(-c2ccc(F)cc2)nn(-c2ccc(C(F)(F)F)c(O)c2)c1=O] | 1 nM-100 nM | 449.18 |
| 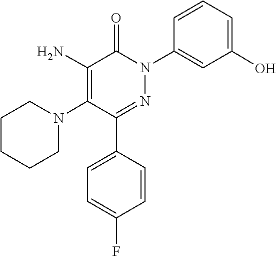 [SMILES: Nc1c(N2CCCCC2)c(-c2ccc(F)cc2)nn(-c2cccc(O)c2)c1=O] | 1 nM-100 nM. | 381.04 |
| 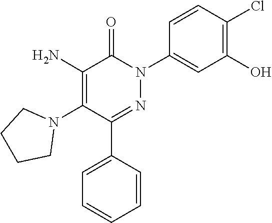 [SMILES: Nc1c(N2CCCC2)c(-c2ccccc2)nn(-c2ccc(Cl)c(O)c2)c1=O] | 1 nM-100 nM | 383.13 |
| 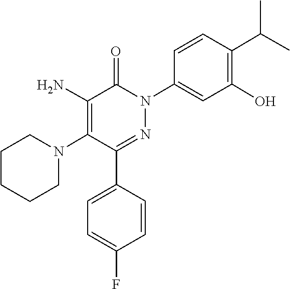 [SMILES: CC(C)c1ccc(-n2nc(-c3ccc(F)cc3)c(N3CCCCC3)c(N)c2=O)cc1O] | 1 nM-100 nM | 423.31 |
| 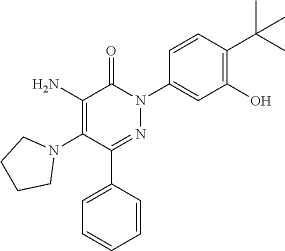 [SMILES: CC(C)(C)c1ccc(-n2nc(-c3ccccc3)c(N3CCCC3)c(N)c2=O)cc1O] | 1 nM-100 nM | 405.23 |
| 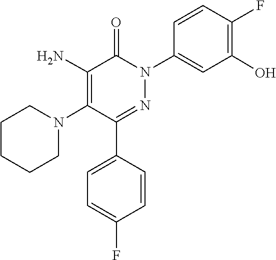 [SMILES: Nc1c(N2CCCCC2)c(-c2ccc(F)cc2)nn(-c2ccc(F)c(O)c2)c1=O] | 1 nM-100 nM | 399.18 |
| 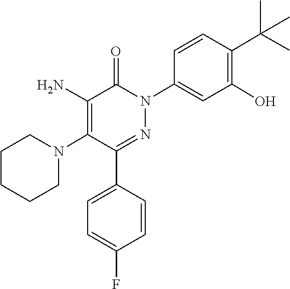 [SMILES: CC(C)(C)c1ccc(-n2nc(-c3ccc(F)cc3)c(N3CCCCC3)c(N)c2=O)cc1O] | 1 nM-100 nM | 437.23 |
| 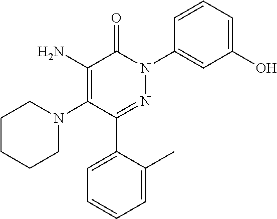 [SMILES: Cc1ccccc1-c1nn(-c2cccc(O)c2)c(=O)c(N)c1N1CCCCC1] | 1 nM-100 nM | 377.20 |
| 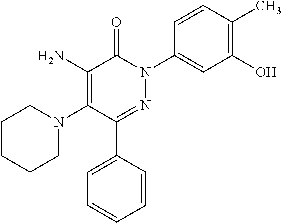 [SMILES: Cc1ccc(-n2nc(-c3ccccc3)c(N3CCCCC3)c(N)c2=O)cc1O] | 1 nM-100 nM | 377.23 |
| 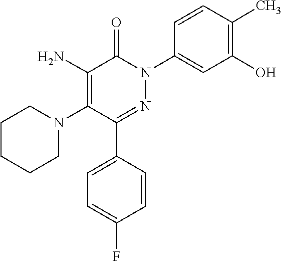 [SMILES: Cc1ccc(-n2nc(-c3ccc(F)cc3)c(N3CCCCC3)c(N)c2=O)cc1O] | 1 nM-100 nM | 395.19 |
| 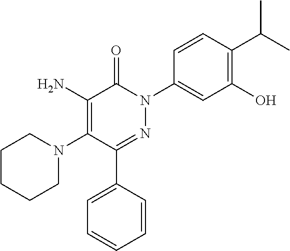 [SMILES: CC(C)c1ccc(-n2nc(-c3ccccc3)c(N3CCCCC3)c(N)c2=O)cc1O] | 1 nM-100 nM | 405.19 |
| 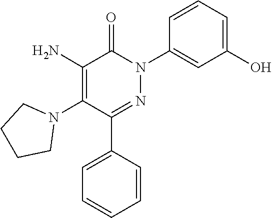 [SMILES: Nc1c(N2CCCC2)c(-c2ccccc2)nn(-c2cccc(O)c2)c1=O] | 1 nM-100 nM | 349.09 |
| 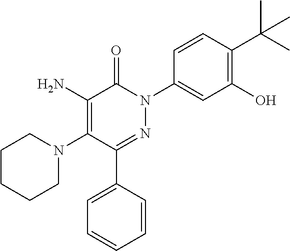 [SMILES: CC(C)(C)c1ccc(-n2nc(-c3ccccc3)c(N3CCCCC3)c(N)c2=O)cc1O] | 1 nM-100 nM | 419.23 |
| 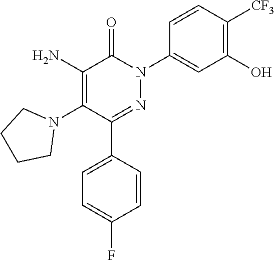 [SMILES: Nc1c(N2CCCC2)c(-c2ccc(F)cc2)nn(-c2ccc(C(F)(F)F)c(O)c2)c1=O] | 1 nM-100 nM | 435.14 |
| 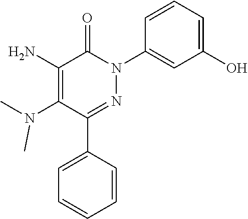 [SMILES: CN(C)c1c(-c2ccccc2)nn(-c2cccc(O)c2)c(=O)c1N] | 1 nM-100 nM | 323.10 |
| 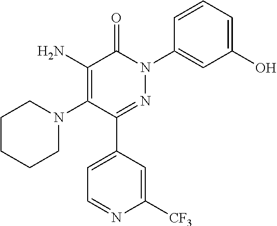 [SMILES: Nc1c(N2CCCCC2)c(-c2ccnc(C(F)(F)F)c2)nn(-c2cccc(O)c2)c1=O] | 1 nM-100 nM | 432.19 |
| 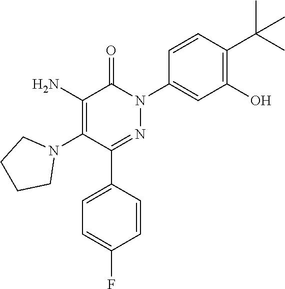 [SMILES: CC(C)(C)c1ccc(-n2nc(-c3ccc(F)cc3)c(N3CCCC3)c(N)c2=O)cc1O] | 1 nM-100 nM | 423.23 |
| 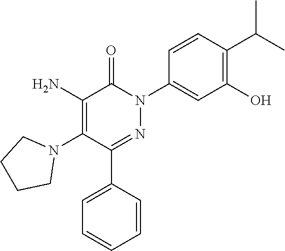 [SMILES: CC(C)c1ccc(-n2nc(-c3ccccc3)c(N3CCCC3)c(N)c2=O)cc1O] | 1 nM-100 nM | 391.24 |
| 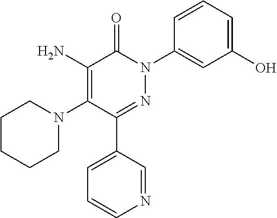 [SMILES: Nc1c(N2CCCCC2)c(-c2cccnc2)nn(-c2cccc(O)c2)c1=O] | 1 nM-100 nM | 364.19 |
| 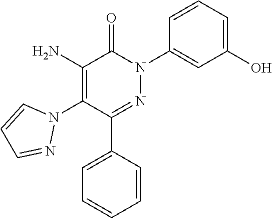 [SMILES: Nc1c(-n2cccn2)c(-c2ccccc2)nn(-c2cccc(O)c2)c1=O] | 1 nM-100 nM | 346.16 |
| 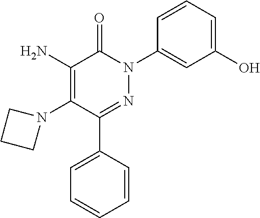 [SMILES: Nc1c(N2CCC2)c(-c2ccccc2)nn(-c2cccc(O)c2)c1=O] | 1 nM-100 nM | 335.35 |
| 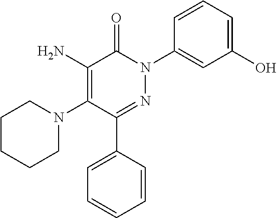 [SMILES: Nc1c(N2CCCCC2)c(-c2ccccc2)nn(-c2cccc(O)c2)c1=O] | 1 nM-100 nM | 363.11 |
| 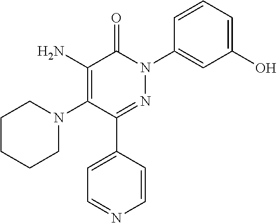 [SMILES: Nc1c(N2CCCCC2)c(-c2ccncc2)nn(-c2cccc(O)c2)c1=O] | 1 nM-100 nM | 364.20 |
| 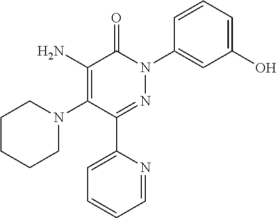 [SMILES: Nc1c(N2CCCCC2)c(-c2ccccn2)nn(-c2cccc(O)c2)c1=O] | 100 nM-1 μM | 364.13 |
| 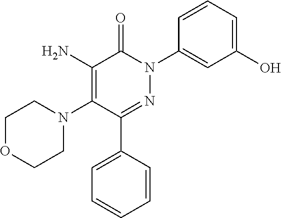 [SMILES: Nc1c(N2CCOCC2)c(-c2ccccc2)nn(-c2cccc(O)c2)c1=O] | 100 nM-1 μM | 365.31 |
| 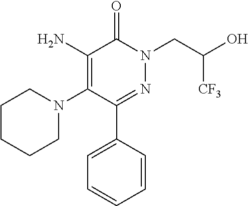 [SMILES: Nc1c(N2CCCCC2)c(-c2ccccc2)nn(CC(O)C(F)(F)F)c1=O] | 100 nM-1 μM | 383.16 |
| 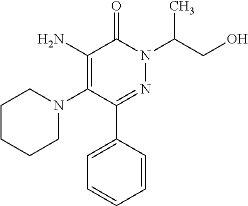 [SMILES: CC(CO)n1nc(-c2ccccc2)c(N2CCCCC2)c(N)c1=O] | 100 nM-1 μM | 329.15 |
| 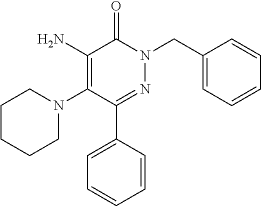 [SMILES: Nc1c(N2CCCCC2)c(-c2ccccc2)nn(Cc2ccccc2)c1=O] | 100 nM-1 μM | 360.9 |
| 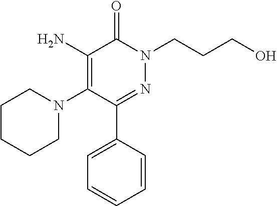 [SMILES: Nc1c(N2CCCCC2)c(-c2ccccc2)nn(CCCO)c1=O] | 1 μM-10 μM | 329.17 |
| 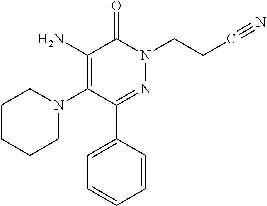 [SMILES: N#CCCn1nc(-c2ccccc2)c(N2CCCCC2)c(N)c1=O] | 1 μM-10 μM | 324.37 |

1. A compound of the Formula (I) 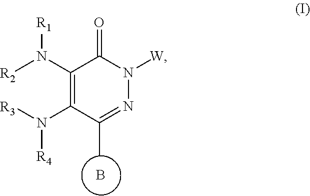
wherein
R₁ and R₂ are independently or simultaneously H or (C₁-C₆)-alkyl;
R₃ and R₄ are independently or simultaneously H or (C₁-C₆)-alkyl, or
R₃ and R₄ are joined together, with the nitrogen atom to which they are attached, to form a (C₅-C₆)-heteroaryl or (C₄-C₆)-heterocycloalkyl, each of which is optionally substituted with halo, OH, (C₁-C₆)-alkyl, or halogenated-(C₁-C₆)-alkyl;
Ring B is (C₆-C₁₀)-aryl or (C₅-C₁₀)-heteroaryl, each of which is optionally substituted with one or more of halo, OH, (C₁-C₆)-alkyl, halogenated-(C₁-C₆)-alkyl, (C₃-C₆)-cycloalkyl, or halogenated-(C₃-C₆)-cycloalkyl;
W is (C₁-C₆)-alkyl or —(C₀-C₆)-alkylene-(C₆-C₁₀)-aryl, each of which is optionally substituted with one or more of halo, OH, CN, (C₁-C₆)-alkyl, halogenated-(C₁-C₆)-alkyl, (C₁-C₆)-alkoxy, (C₃-C₆)-cycloalkyl, or halogenated-(C₃-C₆)-cycloalkyl;
or any pharmaceutically acceptable salt, stereoisomer or solvate thereof.

2. The compound of the Formula (I) according to claim 1, wherein R₁ and R₂ are independently or simultaneously H or (C₁-C₃)-alkyl.

3. (canceled)

4. The compound of the Formula (I) according to claim 1, wherein R₃ and R₄ are independently or simultaneously H or (C₁-C₃)-alkyl, or R₃ and R₄ are joined together, with the nitrogen atom to which they are attached, to form a (C₅)-heteroaryl or (C₄-C₆)-heterocycloalkyl, each of which is optionally substituted with halo, OH, (C₁-C₃)-alkyl, or halogenated-(C₁-C₃)-alkyl.

5. The compound of the Formula (I) according to claim 4, wherein R₃ and R₄ are independently or simultaneously H or CH₃.

6. The compound of the Formula (I) according to claim 4, wherein R₃ and R₄ are joined together, with the nitrogen atom to which they are attached, to form optionally substituted piperidinyl, pyrrolidinyl, morpholinyl, azetidinyl, or pyrazolyl.

7. The compound of the Formula (I) according to claim 1, wherein Ring B is (C₆)-aryl or (C₅-C₆)-heteroaryl, each of which is optionally substituted with one or more of halo, OH, (C₁-C₆)-alkyl, halogenated-(C₁-C₆)-alkyl, (C₃-C₆)-cycloalkyl, or halogenated-(C₃-C₆)-cycloalkyl.

8. The compound of the Formula (I) according to claim 7, wherein Ring B is phenyl or pyridinyl, optionally substituted with one or more of halo, (C₁-C₆)-alkyl, or halogenated-(C₁-C₆)-alkyl.

9. The compound of the Formula (I) according to claim 8, wherein Ring B is phenyl or pyridinyl, optionally substituted with one or more of fluoro or trifluoro-methyl.

10. The compound of the Formula (I) according to claim 9, wherein Ring B has the following structure:

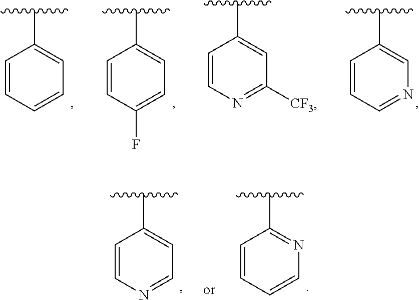

11. The compound of the Formula (I) according to claim 1, wherein W is (C₁-C₄)-alkyl or —(C₀-C₄)-alkylene-(C₆-C₁₀)-aryl, each of which is optionally substituted with halo, OH, CN, (C₁-C₄)-alkyl, halogenated-(C₁-C₄)-alkyl, (C₁-C₄)-alkoxy, (C₃-C₆)-cycloalkyl, or halogenated-(C₃-C₆)-cycloalkyl.

12. The compound of the Formula (I) according to claim 10, wherein W is (C₁-C₄)-alkyl or —(C₀-C₁)-alkylene-phenyl, each of which is optionally substituted with one or more of halo, OH, CN, (C₁-C₄)-alkyl, halogenated-(C₁-C₄)-alkyl, or (C₁-C₄)-alkoxy.

13. The compound of the Formula (I) according to claim 11, wherein W has the following structure

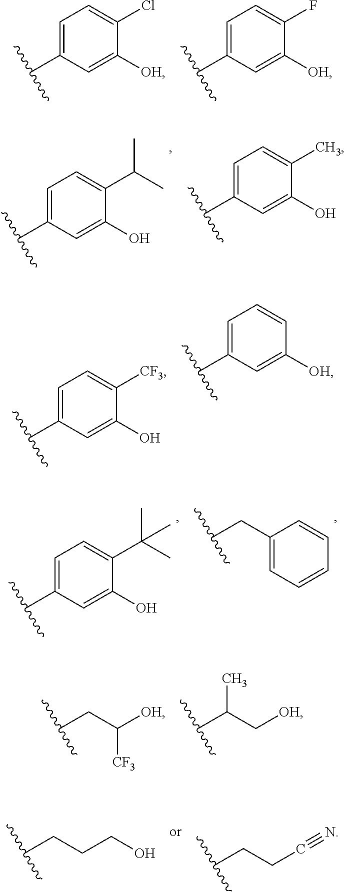

14. The compound of the Formula (I) according to claim 1, wherein the compound of Formula (I) is

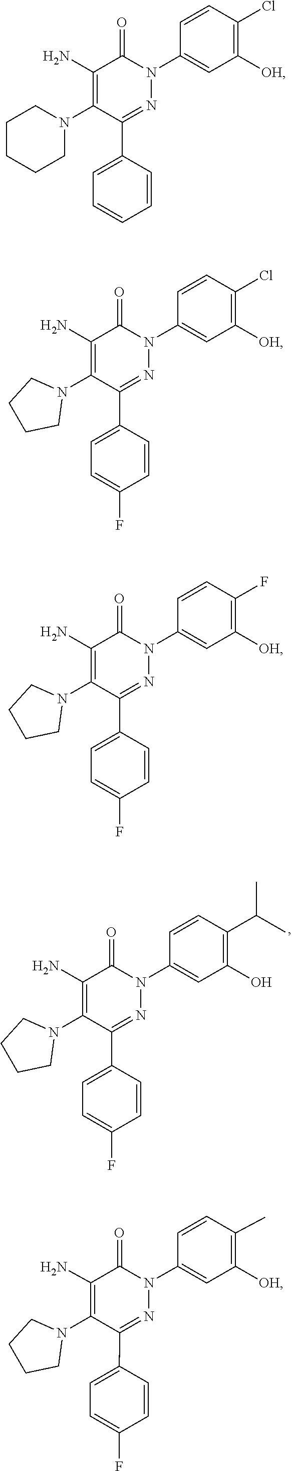 [SMILES: Nc1c(N2CCCCC2)c(-c2ccccc2)nn(-c2ccc(Cl)c(O)c2)c1=O ; Nc1c(N2CCCC2)c(-c2ccc(F)cc2)nn(-c2ccc(Cl)c(O)c2)c1=O ; Nc1c(N2CCCC2)c(-c2ccc(F)cc2)nn(-c2ccc(F)c(O)c2)c1=O ; CC(C)c1ccc(-n2nc(-c3ccc(F)cc3)c(N3CCCC3)c(N)c2=O)cc1O ; Cc1ccc(-n2nc(-c3ccc(F)cc3)c(N3CCCC3)c(N)c2=O)cc1O]

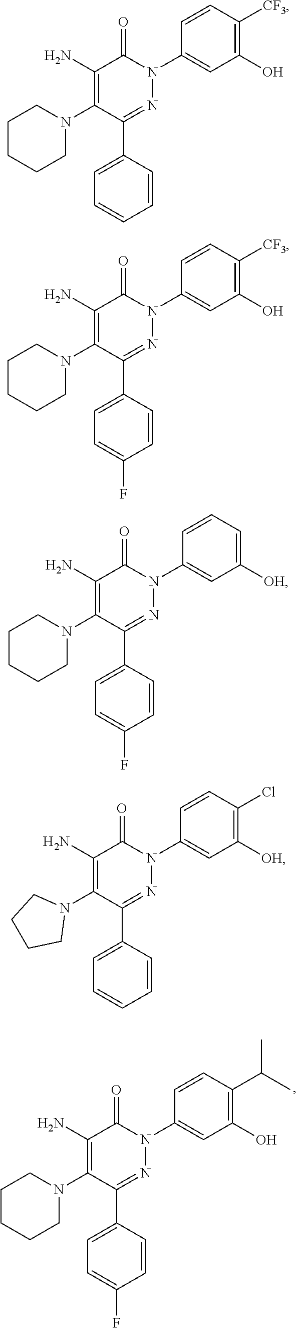

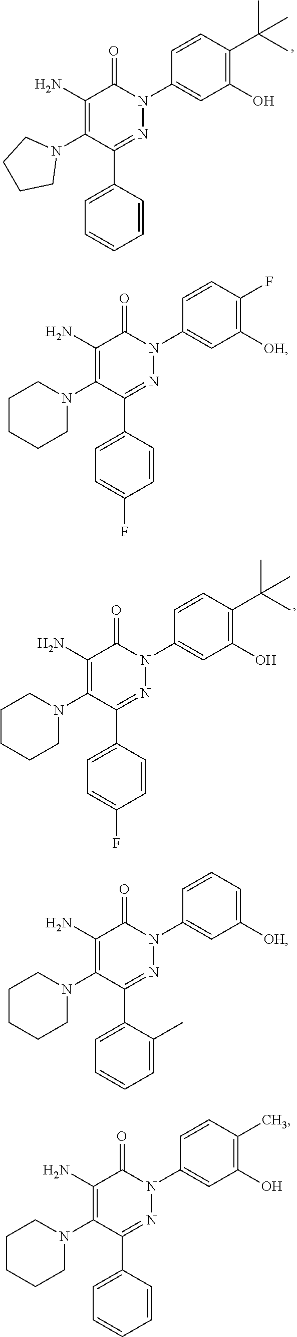

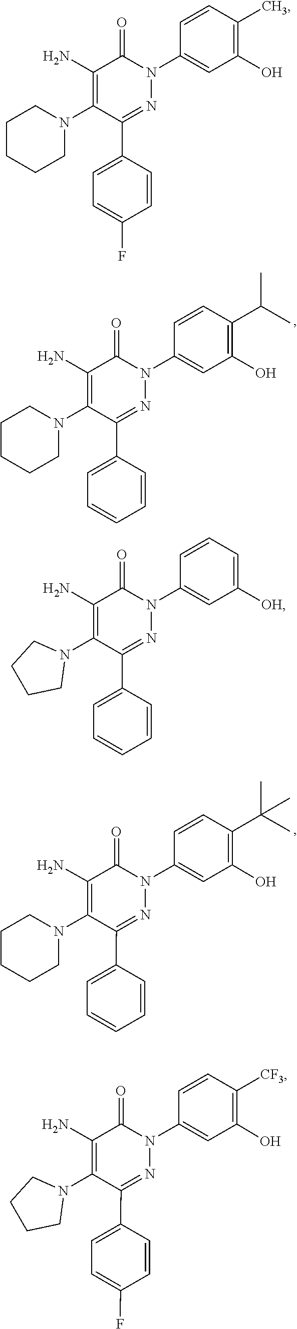

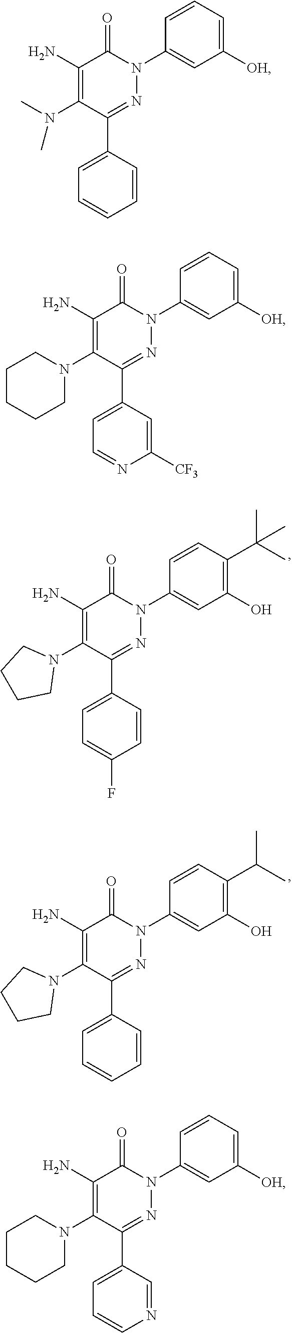 [SMILES: CN(C)c1c(-c2ccccc2)nn(-c2cccc(O)c2)c(=O)c1N ; Nc1c(N2CCCCC2)c(-c2ccnc(C(F)(F)F)c2)nn(-c2cccc(O)c2)c1=O ; CC(C)(C)c1ccc(-n2nc(-c3ccc(F)cc3)c(N3CCCC3)c(N)c2=O)cc1O ; CC(C)c1ccc(-n2nc(-c3ccccc3)c(N3CCCC3)c(N)c2=O)cc1O ; Nc1c(N2CCCCC2)c(-c2cccnc2)nn(-c2cccc(O)c2)c1=O]

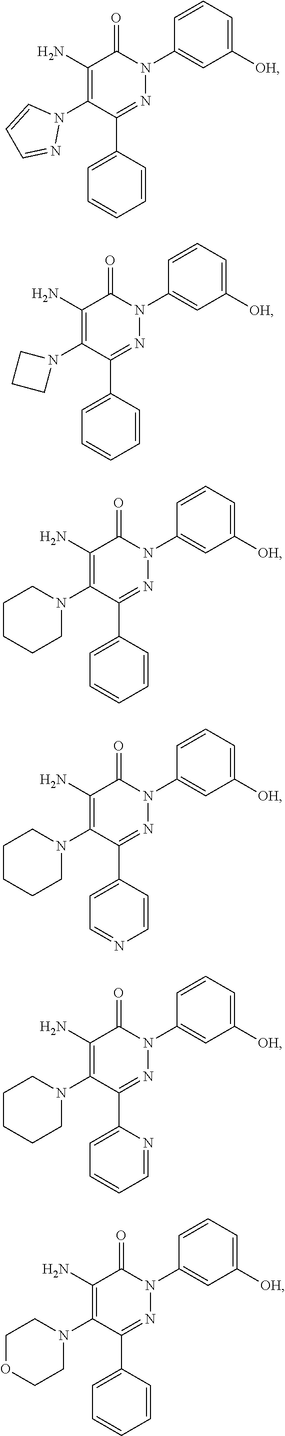

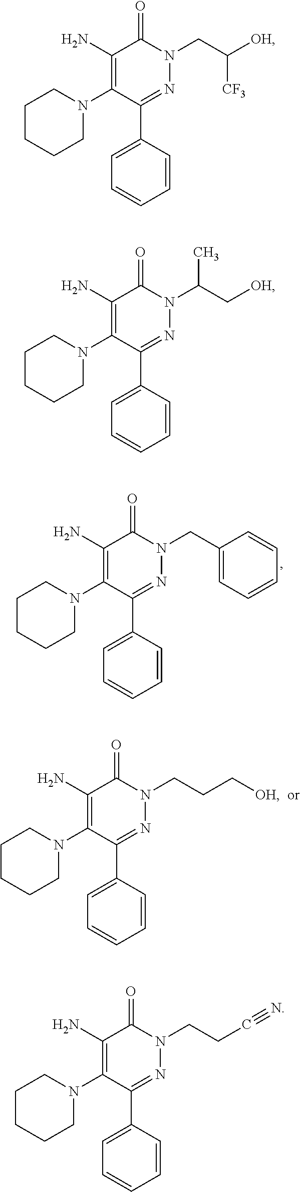

15. The compound of the Formula (I) according to claim 14, wherein the compound is

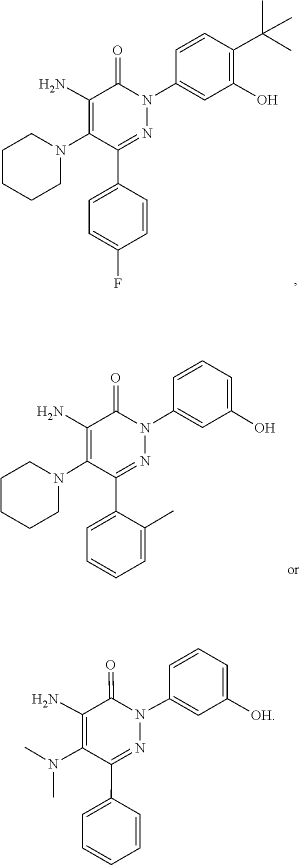

16. The compound of the Formula (I) according to claim 15, wherein the compound is

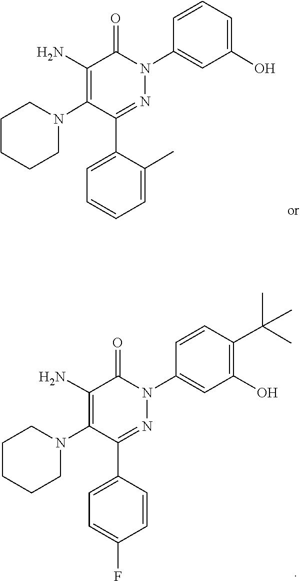 [SMILES: Cc1ccccc1-c1nn(-c2cccc(O)c2)c(=O)c(N)c1N1CCCCC1 ; CC(C)(C)c1ccc(-n2nc(-c3ccc(F)cc3)c(N3CCCCC3)c(N)c2=O)cc1O]

17. A method for the treatment of a disease associated with mis-folded or mis-shaped proteins comprising administering a therapeutically effective amount of a compound of Formula (I) according to claim 1 to a patient in need thereof.

18. The method according to claim 17, wherein the disease is cystic fibrosis, cancer, Long QT syndrome, chronic obstructive pulmonary disorder or Dravet syndrome (epilepsy).

19. The method according to claim 18, wherein the disease is cystic fibrosis or COPD.

20. The method according to claim 19, wherein the cystic fibrosis is a result of the ΔF508 mutation in the CFTR protein.

21. (canceled)

22. A pharmaceutical composition comprising a compound of Formula (I) according to claim 1 and a pharmaceutically acceptable excipient.
